(19) United States
(12) Patent Application Publication
(10) Pub. No.: US 2024/0029886 A1
(43) Pub. Date: Jan. 25, 2024

(54) Method For Training And Quantifying Specific Motor Skills And Cognitive Processes In Persons By Analysing Oculomotor Patterns W Using A 3-D Virtual Reality Device With Embedded Eye-Tracking Technology, Specific Visual Stimuli, Sensors To Show The Movement Of Limbs

(71) Applicant: VIEWMIND, INC., New York, NY (US)

(72) Inventor: Gerardo ABEL FERNANDEZ, Bahia Blanca (AR)

(21) Appl. No.: 18/237,111

(22) Filed: Aug. 23, 2023

Related U.S. Application Data
(63) Continuation-in-part of application No. 18/227,577, filed on Jul. 28, 2023.
Application No. 18/227,577 is a continuation-in-part of application No. 18/217,688, filed on Jul. 3, 2023.
Application No. 18/217,688 is a continuation of application No. 16/768,738, filed on Jun. 1, 2020, now Pat. No. 11,694,803.
(60) Provisional application No. 63/393,025, filed on Jul. 28, 2022.
(60) Provisional application No. 63/373,228, filed on Aug. 23, 2022.
(60) Provisional application No. 62/617,622, filed on Jan. 16, 2018.
(60) Provisional application No. 62/592,517, filed on Nov. 30, 2017.

(51) Int. Cl. G16H 50/20; A61B 5/16; G16H 40/63; G06N 20/00; A61B 3/00; A61B 3/11; A61B 3/113; A61B 5/00

(52) U.S. Cl. CPC G16H 50/20; A61B 5/163; G16H 40/63; G06N 20/00; A61B 3/0025; A61B 3/0041; A61B 3/112; A61B 3/113; A61B 5/162; A61B 5/168; A61B 5/4058; A61B 5/4082; A61B 5/4088; A61B 5/7267

(57) ABSTRACT

Systems and methods combine virtual reality (VR), eye-tracking (ET), and motion sensors on limbs such as hands and feet to evaluate the changes of cognitive and motor abilities in both healthy and non-healthy persons using well-defined exercises. The application of VR and ET in cognitive exercises with motion sensors can improve the efficacy of the intervention and the ability to quantify cognitive and motor capabilities, enhancing the effectiveness of the training on a person.

### CROSS REFERENCE TO RELATED APPLICATION

This application is a continuation in part of U.S. application Ser. No. 18/227,577, filed Jul. 28, 2023, which is a continuation in part of U.S. Ser. No. 18/217,688, filed 3 Jul. 2023, which is a continuation of U.S. application Ser. No. 16/768,738, filed 1 Jun. 2020, now U.S. Pat. No. 11,694,803, which is a 371 National Stage of PCT/IL2018/051316, filed 30 Nov. 2018. This application also claims the benefit of U.S. Provisional Application No. 63/373,228, filed Aug. 23, 2022 and U.S. Provisional Application No. 63/393,025, filed 28 Jul. 2022. The contents of the above applications are incorporated hereinby reference.

### BACKGROUND

Eye tracking systems have been used as a diagnostic tool. For instance, co-pending U.S. application Ser. No. 18/217,688 [Docket No. 9901/1c1], which is incorporated by reference herein in its entirety, shows a system for detecting one or more neurological disorders in a subject by measuring eye movements. Examples of neurological disorders that may be detected include Multiple sclerosis (MS), attention deficit-hyperactive disorder (ADHD), Parkinson disorder (PD), Alzheimer disease (AD), etc.

In addition, virtual Reality (VR) has been used for training cognitive abilities. Human cognitive ability can be roughly classified into working memory, attention, perception, reasoning and judgment, decision-making and so on. However, VR studies are mostly performed by collecting partial data related to behavioral feedback e.g., What is the number of correct responses when performing an activity? or what is the total time needed to complete a task? There is a need for a more comprehensive and in-depth VR studies to assess, train and improve cognitive abilities.

### SUMMARY OF THE INVENTION

It is the object of the present invention to provide a system for detecting one or more neurological disorders in a subject by measuring eye movements; the measuring of eye movements performed while the subject is reading; the system comprising
a. an eye tracker [10], configured to monitor eye movements of a subject [5] while the subject [5] is reading a text [15];
b. a processor [20], configured to receive data from the eye tracker while the subject [5] is reading the text [15]; and
c. a display means [40] configured to display a test report [50] received from the processor [20]; wherein the processor is further configured to analyze the eye-tracking data for evidence of one or more neurological disorders or general cognitive performance and to report, in the test report [50], a detection of one or more neurological disorders or a measure of cognitive performance of the subject [5].

It is another object of the present invention as described that the processor is further configured, upon receiving the eye-tracking data from the eye tracker, to:
a. count a total number of ocular fixations of a subject while reading the text; and
b. if the total number of ocular fixations of a subject when reading is higher than for a control group, then report in the test report that a compromise in attentional processes is detected.

It is another object of the present invention as described above, wherein the processor is further configured, upon receiving the eye-tracking data from the eye tracker, to:
a. count a number of forward ocular fixations of the subject while reading the text; and
b. if the number of forward ocular fixations of the subject is lower than for the control group; and the number of ocular fixations of a subject when reading is higher than for the control group, then report in the test report [50] that a compromise in working memory is detected.

It is another object of the present invention as described above, wherein the processor is further configured, upon receiving the eye-tracking data from the eye tracker, to:
a. count a number of words that the subject fixated on only once while reading the text; and
b. if the number of words that the subject fixated on only once is lower than for the control group, then report in the test report that a compromise in retrieval memory is detected.

It is another object of the present invention as described above, wherein the processor is further configured, upon receiving the eye-tracking data from the eye tracker, to:
a. count a number of multiple ocular fixations of the subject while reading the text; and
b. if the number of multiple ocular fixations is higher than for the control group, then report in the test report that a compromise in executive processes is detected.

It is another object of the present invention to detecting one or more neurological disorders in a subject by measuring eye movements, wherein the processor is further configured, upon receiving the eye-tracking data from the eye tracker, to
a. compute an average saccade amplitude from one ocular fixation to a next ocular fixation; and
b. if the average saccade amplitude is lower than for the control group, then report in the test report that a compromise in executive processes is detected.

It is another object of the present invention as described above, further comprising a means [17] for measuring a pupil diameter of the subject, wherein the processor is further configured to
a. track the pupil diameter of the subject reading the text; and
b. if the pupil diameter of the subject does not show a reduction as advancing in reading the text, then report in the test report that that a compromise in executive processes is detected.

It is the object of the present invention to provide a system for detecting one or more neurological disorders and to check cognitive performance in a subject by measuring eye movements and pupil behavior and applying an intelligent algorithm; the measuring of eye movements performed while the subject is reading; the system comprising
a. an eye tracker [10], the eye tracker configured to monitor eye movements and pupil behavior of a subject [5] while the subject [5] is reading a text [15];
b. a processor [20], the processor configured to receive data from the eye tracker [10] while the subject [5] is reading the text [15];
c. an intelligent algorithm for learning, identifying, typifying and classifying eye movements features in pathologies and within pathologies; and
d. a display means [40], the display configured to display the output of the intelligent algorithm on a test report [50] received from the processor [20];
   wherein the processor [20] is further configured to analyze and modeling the eye-tracking data for evidence of one or more neurological disorders and from cognitive performance and to report, in the test report [50], a detection and classification of the one or more neurological disorders of the subject [5] both, between and within pathologies.

It is another object of the present invention as described above, wherein the processor is further configured, upon receiving the eye-tracking data from the eye tracker, to identify and classifying eye movement features and pupil behavior during reading the text providing an output of the classifier for reporting in the test report a subject's cognitive performance and/or pathological classification (i.e, the pathology that correspond to the subject because his/her eye movement features); and a value within the pathology (i.e., the level of cognitive, behavioral and biological compromise that the subject shows within a particular pathology).

It is another object of the present invention as described above, wherein the intelligent algorithm is configured to read at least one input, the input selected from a group consisting of:
a. Index of total number of ocular fixations of a subject while reading the text.
b. Index of forward ocular fixations of the subject while reading the text.
c. Index of words that the subject fixated on only once while reading the text
d. Index of multiple ocular fixations of the subject while reading the text
e. Average saccade amplitude from one ocular fixation to a next ocular fixation
f. Pupil diameter of the subject reading the text
g. Index of blinks coming from the left eye, the right eye or from both eyes.
h. Microsaccades' Factors of Form (FF):
i. HEWI: shows the micro-saccade's height/width relationship.
ii. AREA: shows the area of the rectangle in which the micro-saccade is inscribed.
iii. LONG: is the longitude of the horizontal-vertical plane trajectory of the micro-saccade. iv. ANG: is the sum of all the angles in the plane horizontal-vertical plane of the micro-saccade.
v. AANG: is the sum of all the absolute values of angles in radians in the plane horizontal-vertical plane of the micro-saccade. These last two FF gives an estimation of the micro-saccadic trajectory regularity.
vi. MOD and THETA: are the modulus and the angle of the polar coordinates of the sum of the cartesian coordinates. They give a spatial orientation of the micro-saccade relative to the median of the fixation.
vii. TIME: is the time duration in milliseconds of the micro-saccade.
viii. VMIN and VMAX: are the minimum and maximum velocities of the microsaccades in degrees per second.
ix. Micro-saccade rate: is the instantaneous rate in each time bin.
x. Directional congruency: is the congruency between the micro-saccade direction ant the location of the stimulus.
i. Eye position coming from the left eye, the right eye or from both eyes (i.e., abscissa and ordinate coordinate) during reading the text.
j. Fixation sequence (i.e., ocular behavior) during reading the text. The sequence will be available from images, from matrices, etc.
k. Distance of separation between ocular fixations during reading the text.
l. Filia information of the subject (i.e., age; years of education; sex; ethnic group; occupation; hours per week of physical activity).
m. Total reading time (i.e., the time that the subject spent when reading the text).

It is the object of the present invention to provide a method [300] for evaluating compromises in neurological functions associated with Multiple Sclerosis [MS], the method comprising
a. providing a system for evaluating compromises in neurological functions associated with MS [305];
b. requesting a subject to fixate on a reference target of a chart [310];
c. for a number of repetitions, presenting a stimulus image in one of the zones to the subject [315]; the subject is requested to remember which zone each stimulus image appeared and in what order;
d. presenting to the subject a cue corresponding to one of the presented stimulus images [320];
e. measuring a saccade of the subject [325] in response to the step of presenting a cue; the subject is requested to look at the zone in which the stimulus image was the presented corresponding to the cue;
f. repeating steps of presenting a cue and measuring a saccade [330];
g. repeating steps b-f for a number of trials [335];
h. calculating one or more of:
i. a WM effect [340] (i.e. WM effect is a measure that increases when WM demand increases. For each cue number, the WM effect is represented by the ratio between the number of errors reported by the subject through all the trials, and the number of trials); and
ii. an average saccadic latency [345], saccadic latency defined as an amount of time for the subject to initiate a saccade to the zone; and
i. reporting one or more of:
i. a degree of compromise in working memory [350], with increased WM effect; and
ii. a degree of compromise in executive processes [355], with increased saccadic latency;
j. wherein the method further comprises additional steps comprising measurements performed during the step of presenting a stimulus image [315], during which the subject is further requested to look at the stimulus image; the measurements comprising measuring one or more of
i. an amplitude of pupillary dilatation of the subject [360];
ii. a number of fixations made by the subject on the stimulus image [365]; and
iii. a gaze duration by the subject on the stimulus image [370]; and
k. the additional steps further comprising calculating and reporting one or more of
i. a degree of compromise of subcortical processes [375], with increased the amplitude ofpupillary dilatation;
ii. a degree of compromise of executive processes [380], with increased the number of fixations; and
iii. a degree of compromise of executive processes and working memory [385], with increased the gaze duration.

It is another object of the present invention as described above, wherein the reference target is at a central position of the chart and the plurality of zones are disposed around the reference target.

It is another object of the present invention as described above, wherein the cue is disposed at a position of the reference target.

It is another object of the present invention as described above, wherein the errors defined as eye movement towards a location other than the correct zone and/or no saccade initiated within a time limit.

It is another object of the present invention as described above, wherein a cue corresponding to a first presented stimulus is excluded from the presented cue numbers.

It is another object of the present invention as described above, wherein a saccade is included in the step of calculating the WM effect and the saccadic latency only if the saccade is initiated more than a minimum saccade latency after the step of presenting the cue number.

It is another object of the present invention as described above, wherein the saccade is excluded from calculating WM if: no saccade to one of the zones is made within a time limit, failing to maintain the fixation on the reference target before onset of a saccade to one of the angular zones, and blinking causing eye motion to be indeterminate
a. It is the object of the present invention to provide a system for detecting one or more neurological disorders in a subject by measuring eye movements; the measuring of eye movements performed while the subject is carrying out the visual test; the system comprising
an eye tracker [10], configured to monitor eye movements of a subject [5] while the subject [5] is carrying out the visual test [15];
b. requesting a subject to fixate sequentially on targets that are part of a group of targets (e.g., point) presented together in the same picture (i.e., labyrinth or maze) [605];
c. requesting a subject to fixate only one target each time until finishing visualizing all the targets through the picture following the labyrinth or maze direction (i.e., entering from the bottom and exiting through the top of said labyrinth or maze) [610].
d. a processor [20], configured to receive data from the eye tracker [10] while the subject [5] is carrying out the visual test [15]; and
e. a display means [40] configured to display a test report [50] received from the processor [20];
   wherein the processor [20] is further configured to analyze the eye-tracking data for evidence of neurological and attentional disorders and to report, in the test report [50], a detection of the one or more neurological and attentional disorder of the subject [5].

It is another object of the present invention as described above, wherein the processor is further configured, upon receiving the eye-tracking data from the eye tracker while the subject is visualizing, recognizing, maintaining, controlling, inhibiting and sequencing targets, to:
a. count a total number of ocular fixations of a subject [615] while performing the visual test; and
b. if the total number of ocular fixations of a subject when visualizing targets is higher than for a control group, then report in the test report that a compromise in attentional processes is detected.

It is another object of the present invention as described above, wherein the processor is further configured, upon receiving the eye-tracking data from the eye tracker, to:
a. measure the saccade average speed [620] while the subject is shifting from one target to the other; and
b. if the saccade average speed [620] of the subject is lower than for the control group; then report in the test report [50] that a compromise in executive functions is detected.

It is another object of the present invention as described above, wherein the processor is further configured, upon receiving the eye-tracking data from the eye tracker, to:
a. count a number of correct target recognitions [625]; and
b. if the number of correct target recognitions [625] that the subject is lower than for the control group, then report in the test report that a compromise in working memory is detected.

It is another object of the present invention as described above, wherein the processor is further configured, upon receiving the eye-tracking data from the eye tracker, to:
a. compute an average saccade amplitude [630]; and
b. if the average saccade amplitude [630] is lower than for the control group, then report in the test report that a compromise in executive processes is detected.

It is another object of the present invention as described above, wherein the processor is further configured, upon receiving the eye-tracking data from the eye tracker, to:
a. the total time spent to perform the visual test [635]; and
b. if the total time spent to perform the visual test [635] is higher than for the control group, then report in the test report that a compromise in attentional processes is detected.

It is another object of the present invention as described above, further comprising a means [17] for measuring a pupil diameter of the subject, wherein the processor is further configured to:
a. track the pupil diameter of the subject [640] performing the visual test; and
b. if the pupil diameter of the subject [640] does not show an increase when advancing in performing the task, then report in the test report that that a compromise in attentional processes is detected.

It is another object of the present invention as described above, further comprising a means [17] for measuring a pupil diameter of the subject, wherein the processor is further configured for calculating fixation durations on targets of person while performing the visual test, if the fixation duration of the subject [645] while fixating on targets is lower than for the control group, then report in the test report that that a compromise in attentional and executive processes is detected.

It is the object of the present invention to provide a method [400] for detecting the presence of one or more neurological disorders or for measuring general cognitive performance in a subject by measuring eye movements of the subject; the measuring of eye movements performed while the subject is reading [405]; the method comprising steps of:
a. providing the system for detecting one or more neurological disorders of claim 1 or claim 18;
b. receiving eye-tracking data and/or pupil diameter data of a subject while the subject is reading a text [415];
   wherein the method further comprises steps of analyzing the eye-tracking data and/or pupil diameter data for evidence of one or more neurological disorders [417] and displaying a report of a detection of the neurological disorder(s) [499].

It is another object of the present invention as described above, further comprising steps of:
a. counting a total number of ocular fixations of the subject while the subject is reading the text [420]; and
b. if the total number of ocular fixations of the subject while reading the text is higher than for a control group, then reporting that a compromise in attentional processes is detected [460].

It is another object of the present invention as described above, further comprising steps of:
a. counting a total number of ocular fixations of the subject while the subject is reading the text [420];
b. counting a number of forward ocular fixations of the subject while the subject is reading the text [430]; and
c. if the number of forward ocular fixations of the subject while reading the text [430] is lower than for the control group; and the number of ocular fixations of a subject when reading is higher than for the control group, then reporting that a compromise in working memory is detected [470].

It is another object of the present invention as described above, further comprising steps of:
a. counting numbers of ocular fixations by the subject on each word in the text while the subject is reading the text [440];
b. counting a number of the words that the subject fixated on only once while reading the text [445]; and
c. if the number of words that the subject fixated on only once while reading the text [445] is lower than for the control group, then reporting that a compromise in retrieval memory is detected [480].

It is another object of the present invention as described above, further comprising steps of:
a. counting a number of multiple ocular fixations of the subject while reading the text[450]; and
b. if the number of multiple ocular fixations of the subject while the subject is reading the text [450] is higher than for the control group, then reporting that a compromise in executive processes is detected [490].

It is another object of the present invention as described above, further comprising steps of:
a. computing an average saccade amplitude of the subject from one ocular fixation to a next ocular fixation while reading the text [454]; and
b. if the average saccade amplitude of the subject from one ocular fixation to a next ocular fixation while reading the text [454] is lower than for the control group, then reporting in the test report that a compromise in executive processes is detected [491].

It is another object of the present invention as described above, further comprising steps of:
a. tracking a pupil diameter of the subject reading the text [456];
b. if the pupil diameter of the subject reading the text [456] does not show a reduction as advancing in reading the text, then reporting in the test report that a compromise in executive processes is detected [492].

It is the object of the present invention to present a system [100] for detecting a disorder of memory binding function of a subject, the system comprising:
a. an eye tracker [10];
b. a means for measuring pupil diameters;
c. a processor [20], configured to:
i. receive eye-tracking data of a subject [5] from the eye tracker [10];
ii. receive pupil diameter data of the subject [5] from the means for measuring pupil diameters; and
d. a display means [40] configured to display a test report [50] received from the processor [20];
   wherein the processor [20] is further configured to analyze the eye-tracking and pupil diameter data and to report, in the test report [50], a detection of one or more disorders of memory binding function of the subject [5].

It is another object of the present invention as described above, wherein the processor [20] is further configured, upon receiving the eye-tracking data from the eye tracker [10], to:
a. measure one or more gaze durations of the subject [5] on each of one or more targets viewed by the subject [5];
b. calculate an average gaze duration of the targets by the subject [5]; and
c. report in the test report [50] that a compromise in encoding and recognition of targets is detected in the subject [5], if the average gaze duration of the subject [5] is longer than the average gaze duration of a control group.

It is another object of the present invention as described above, wherein the processor [20] is further configured, upon receiving the eye-tracking data from the eye tracker [10], to:
a. count a number of ocular fixations performed by the subject [5] while viewing one or more targets; and
b. report in the test report [50] that a compromise in attentional processes is detected in the subject [5], if the number of ocular fixations performed by the subject [5] while viewing the targets is higher than for a control group.

It is another object of the present invention as described above, wherein the processor [20] is further configured to applying an intelligent algorithm and to:
a. receive a pupil diameter of the subject [5] from the means for measuring pupil diameter, while the subject [5] performs activities requiring lower cognitive effort;
b. receive a pupil diameter of the subject [5] from the means for measuring pupil diameter, while the subject [5] performs activities requiring a stronger cognitive effort; and
c. report in the test report [50] that a compromise in cognitive resources is detected in the subject [5], if the pupil diameter of the subject [5], while performing the activities requiring the stronger cognitive effort, does not show an increase over the pupil diameter of the subject [5] while performing the activities requiring reduced/minimal cognitive effort.

It is another object of the present invention as described above, wherein the processor [20] further reports a result in the test report [50], for the disorder of memory binding function not detected by the system [100] in the subject [5].

It is the object of the present invention to provide a method [500] for detecting a disorder of memory binding function of a subject [505], the method comprising steps of:
a. providing a system of claim 1 or claim 33;
b. Presenting targets [510];
c. Requesting a subjects to fixate on targets and to remember them (Encoding) [515];
d. Presenting an empty screen [520];
e. Presenting targets and requesting a subject to identify if the targets are exactly the same that were viewed before (Recognition). If the targets are exactly the same an answer saying “same” must be given. If are not exactly the same, an answer saying “different must be given. Both answers must be collected using a keyboard or similar support [525]. Repeating steps from [510-525] for a number of trials [530];
f. Repeating steps [510-525] for a number of trials [530];
g. receiving eye-tracking data;
h. viewing by a subject of one or more targets [540];
i. measuring the gaze duration of the subject on each of the targets [545];
j. calculating an average gaze duration of the targets by the subject [550];
k. measuring a pupil diameter of the subject while performing activities requiring lower cognitive effort [555];
l. counting a number of ocular fixations performed by the subject while viewing the targets [560];
m. wherein the method further comprises steps of:
i. reporting that a compromise in a target encoding and recognition process is detected in the subject, if the average gaze duration of the subject is longer than an average gaze duration of a control group [565];
ii. reporting that a compromise in cognitive resources is detected in the subject, if the pupil diameter of the subject while performing the activities requiring a stronger cognitive effort does not show an increase over the pupil diameter of the subject while performing the activities requiring lower cognitive effort [570]; and
iii. reporting that a compromise in attentional processes is detected in the subject, if the number of ocular fixations performed by the subject while viewing the targets is higher than for a control group [575].

It is another object of the present invention as described above, wherein the intelligent algorithm is configured to read at least one input, the input selected from a group consisting of:
a. Total number of ocular fixations of a subject while performing each Binding Task.
b. Binding Evaluation Task, i.e. “Bound Colors” of “Unbound Colors”.
c. Identification Number of Binding Trial.
d. The Correct Behavioral Answer of the trial (i.e., if “same” or “different”).
e. Subject's Behavioral response.
f. Part of the Trial i.e., encoding or retrieval.
g. Pupil diameter of the subject while performing while performing the Binding Evaluation.
h. Number of blinks coming from the left eye, the right eye or from both eyes.
i. Microsaccades; Factors of Form (FF):
i. HEWI: shows the microsaccade's height/width relationship.
ii. AREA: shows the area of the rectangle in which the microsaccade is inscribed. LONG: is the longitude of the horizontal-vertical plane trajectory of the microsaccade.
iv. ANG: is the sum of all the angles in the plane horizontal-vertical plane of the microsaccade.
v. AANG: is the sum of all the absolute values of angles in radians in the plane horizontal-vertical plane of the microsaccade. These las two FF give an estimation of the microsaccadic trajectory regularity.
vi. MOD and THETA: are the modulus and the angle of the polar coordinates of the sum of the cartesian coordinates. They give an spatial orientation of the microsaccade relative to the median of the fixation.
vii. TIME: is the time duration in milliseconds of the microsaccade.
viii. VMIN and VMAX: are the minimum and maximum velocities of the microsaccades in degrees per second.
ix. Microsaccade rate: is the instantaneous rate in each time bin.
x. Directional congruency: is the congruency between the microsaccade direction and the location of the stimulus.
j. Eye position coming from the left eye, the right eye or from both eyes (i.e., abscissa and ordinate coordinate) while performing the Binding Evaluation.
k. Saccade amplitude while processing targets.
l. Fixation sequence (i.e., ocular behavior) during processing targets. The sequence will be available from images, from matrices, etc.
m. Distance between the fixation point of the Right Eye and the Left Eye while performing the Binding Evaluation.
n. Filia information of the subject (i.e., age; years of education; sex; ethnic group; occupation; hours per week of physical activity).
o. Fixation duration while processing targets.
p. Gaze duration while processing targets.
q. Number of fixations on each target.
r. Number of fixations outside each target.
s. Number of fixation on each target.

It is the object of the present invention to provide a method [600] for detecting a neurological and attentional disorders of a subject, the method comprising steps of:
a. providing an eye tracker [10];
b. a means for measuring pupil diameters;
c. a processor [20], configured to:
i. receive eye-tracking data of a subject [5] from the eye tracker [10];
ii. receive pupil diameter data of the subject [5] from the means for measuring pupil diameters; and
iii. a display means [40] configured to display a test report [50] received from the processor[20];
   wherein the processor [20] is further configured to analyze the eye-tracking and pupil diameter data and to report, in the test report [50], a detection of one or more neurological and attentional disorders of the subject [5].

It is another object of the present invention as described above, wherein the processor [20] is further configured, upon receiving the eye-tracking data from the eye tracker [10], to:
a. measure one or more fixation durations of the subject [5] on each of one or more targets viewed by the subject [5];
b. calculate an average saccade amplitude from each target to the other one by the subject [5]; and
c. report in the test report [50] that a compromise in visualizing, recognizing, maintaining, controlling, inhibiting and sequencing of targets is detected in the subject [5], if the average saccade amplitude of the subject [5] is shorter than the average saccade amplitude of a control group.

It is another object of the present invention as described above, wherein the processor [20] is further configured, upon receiving the eye-tracking data from the eye tracker [10], to:
a. count a number of ocular fixations performed by the subject [5] while viewing one or more targets; and
b. report in the test report [50] that a compromise in attentional processes is detected in the subject [5], if the number of ocular fixations performed by the subject [5] while viewing the targets is higher than for a control group.

It is another object of the present invention as described above, wherein the processor [20] is further configured to:
a. receive a pupil diameter of the subject [5] from the means for measuring pupil diameter, while the subject [5] performs activities requiring major attention resources;
b. receive a pupil diameter of the subject [5] from the means for measuring pupil diameter, while the subject [5] performs activities requiring a major attention; and
c. report in the test report [50] that a compromise in cognitive resources is detected in the subject [5], if the pupil diameter of the subject [5], while performing the activities requiring the major attention, does not show an increase over the pupil diameter of the subject [5] while performing the activities requiring minor attention.

It is the object of the present invention to provide a method [600] for detecting a neurological and executive disorder of a subject, the method comprising steps of
a. providing a system as described above;
b. receiving eye-tracking data;
c. viewing by a subject of one or more targets [605-610];
d. calculating an average saccade amplitude of the targets by the subject [630];
e. measuring a pupil diameter of the subject while performing activities requiring major attention [640];
f. measuring a pupil diameter of the subject while performing activities requiring a major attention than the minor attention; and
g. counting a number of ocular fixations performed by the subject while viewing the targets [615];
h. wherein the method further comprises steps of:
i. reporting that a compromise in a target visualizing, recognizing, maintaining, controlling, inhibiting and sequencing process is detected in the subject, if the average saccade amplitude of the subject is shorter than an average saccade amplitude of a control group; ii. reporting that a compromise in cognitive and functional resources is detected in the subject,
if the pupil diameter of the subject while performing the activities requiring a major attention does not show an increase over the pupil diameter of the subject while performing the activities requiring minor attention; and reporting that a compromise in attentional processes is detected in the subject, if the number of ocular fixations performed by the subject while viewing the targets is higher than for a control group.
iii. reporting that a compromise in executive process is detected in the subject, if the average saccade latency (speed) of the subject is shorter than an average saccade latency of a control group;
It is another object of the present invention as described above, wherein the method is configured to report that a compromise in executive process is detected in the subject, if the average saccade duration of the subject is shorter than an average fixation duration of a control group.

It is another object of the present invention as described above, wherein the neurological disorder is selected from the group consisting of Parkinson Disease or Attention Deficit Hyperactive Disorder.

It is another object of the present invention to provide a system and method for evaluating performance, motor skills and cognitive capabilities of a person. The system includes a three-dimensional (3D) virtual reality device configured to establish a 3D virtual reality environment in which a plurality of virtual objects is presented to the person, the objects having at least one feature that differs from one another, the objects moving toward or away from the person with a defined speed, acceleration and direction. The system also includes an eye-tracker configured to measure eye movements of the person while the person is viewing the virtual objects and performing requested tasks, the requested tasks including multiple requests requesting the person to virtually touch specified virtual objects each having one of the specified features. One or more motion sensors are configured to measure limb movements of the person while the person performs the requested tasks. A processor is configured to receive data from the 3D virtual reality device, the eye-tracker and the one or more motion sensors while the person is performing the requested tasks and being further configured to (i) identify selected ones of the measured eye and limb movements that are related to the performance, motor skills and cognitive capabilities of the person; (ii) determine expected eye and limb movements of the person while the person is viewing the virtual objects and performing the requested tasks and comparing the expected eye and limb movements to the selected ones of the measured eye and limb movements to determine deviations therebetween; and (iii) evaluate the performance, motor skills and cognitive capabilities of the person based on the deviations.

### BRIEF DESCRIPTION OF THE FIGURES

FIG. 1 and FIG. 2 show systems for detecting one or more neurological disorders of a subject, according to some embodiments of the invention.

FIGS. 3A and 3B show a method for evaluating compromises in neurological functions associated with MS, according to some embodiments of the invention.

FIGS. 4A and 4B show a method for detecting one or more neurological disorders of a reading subject, according to some embodiments of the invention.

FIG. 5 shows a method for detecting a disorder of memory binding function, according to some embodiments of the invention.

FIG. 5B shows the test results as per the evaluation method of 5A: Corrected recognition during the two experimental conditions in both controls and AD patients (error bars=standard errors of the mean).

FIG. 5C shows the test results as per the evaluation method of 5A: Effect of binding task on gaze duration in control and in Alzheimer Disease (AD) patients during Encoding and Recognition moments. The panel shows the partial effects of LMM (i.e., after removal of other fixed effects and variance components). Shaded areas denote 95% confidence intervals. Gaze duration is plotted on a log scale for correspondence with the LMM.

FIGS. 6A and 6B shows a method for detecting Parkinson Disorder and Attention Deficit Hyperactive Disorder.

FIG. 7 shows the impact of Dimethyl Fumarate on Saccade Amplitude, on a Multiple Sclerosis patient that has been taking the drug for 4 years.

FIG. 8 is a flowchart showing a method for identifying specific alterations in subjects with defined disease analyzing oculomotor patterns when using specific visual stimuli, where a specific drug or treatment would enhance visual processing, cognitive performance and related brain activities.

FIG. 9 shows a conceptual illustration of a system used to evaluate a person's performance by applying a 3-Dimension Virtual Reality (3DVR) environment in combination with an embedded eye-tracking technology (ET) and motions sensors to track the movement of limbs such as hands and feet while the person performs well-defined activities.

FIG. 10 shows an example of how objects may be positioned throughout the virtual environment and how objects and the person's hands may appear.

FIG. 11 is a graphical representation of an example of the density of eye movements recorded from the right and left eye while the person is performing the tasks touching the requested objects, presented as a heat-map.

FIG. 12 is a graphical representation of the density of motor movements recorded of right and left-hand movements while the person is performing the tasks touching the requested objects, presented as a heat-map.

FIG. 13A and FIG. 13B is a flowchart describing one example of the methods described herein for evaluating performance, motor skills and cognitive capabilities of a person.

### DETAILED DESCRIPTION

The term “cognitive effort” reflects the total amount of mental effort that a subject needs to perform a task. In this application, the term “lower cognitive effort” refers to a reduction on working memory demands when performing a task.

In this application, the term “Microsaccades”, also known as “flicks”, are small saccades performed during the fixation periods. They are the largest and fastest of the fixational eye movements. In this application, the term “saccades” relate to quick, simultaneous movement of both eyes between two or more phases of a fixation.

In this application the term “Ocular drift” is the fixational eye movement characterized by a smoother, slower, roaming motion of the eye when fixed on an object.

In this application the term “Ocular microtremors” (OMTs) are small, quick, and synchronized oscillations of the eyes occurring at frequencies in a range of 40 to 100 Hz, although they typically occur at around 90 Hz in the average healthy individual. They are characterized by their high frequency and minuscule amplitude of just a few arcseconds.

In this application the terms “stimulus image” refers to a specific visual pattern or targets presented to the subject in the display. The term “visual task” or “visual test” refers to the activity that performs the subject while processing each stimulus image.

N on-limiting embodiments of the invention are now described in detail.

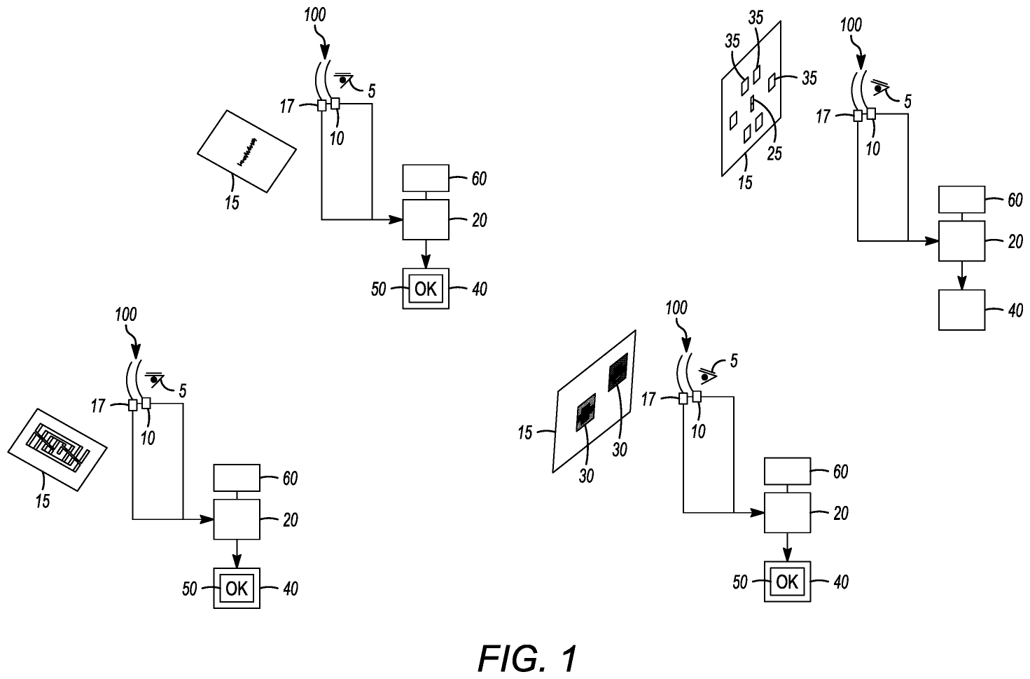

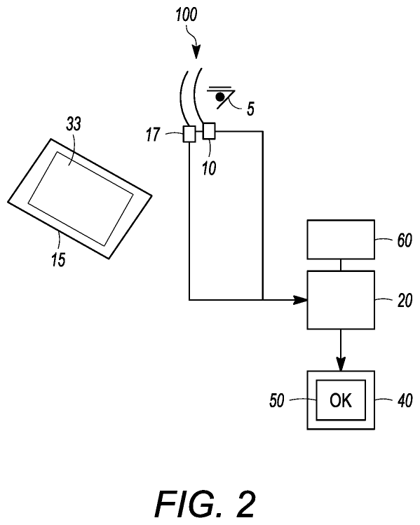

Reference is now made to FIG. 1, showing a system [100] for detecting a neurological disorder or neurological function of a subject [5], according to some embodiments of the invention.

System [100] comprises an eye tracker [10], a means for measuring a pupil diameter[17], a processor [20], and a display means [40].

Eye tracker [10] can be of any type known in the art; for example, an eye-attached tracker, an optical eye tracker, or an electrooculographic eye tracker.

Means for measuring pupil diameter [17] may comprise, for example, a camera configured to acquire an image of the eye and a processing unit for measuring the pupil diameter from the image. Alternatively to a processing unit, means for measuring a pupil diameter [17] can comprise a display of the image with manual measurement made while viewing the display.

Eye tracker [10] and means for measuring a pupil diameter [17] are in communicative connection with processor [20]. The communicative connections can be of any form(s) known in the art, and can be either wired (e.g., USB, parallel port, or similar) or wireless (e.g. WiFi, Bluetooth, or similar).

Processor [20] receives and executes instructions stored in one or more memory media [60], such as RAM, CD/DVD, HDD, flash memory, and/or any suitable medium. The instructions command processor [20] to: 1) receive eye-tracking data from eye tracker[10]; 2) receive pupil diameter data from means [17] of measuring pupil diameter; 3) analyze the eye-tracking and pupil diameter data (further explained herein); 4) report in a test report 50, for display on display means [40], of a detection or non-detection of one or more disorders of memory binding function in subject [5]. Display means [40] can be a monitor, a screen of a mobile device such as a smartphone, a printout, or any suitable means of displaying test report [50]. Processor [20] may store in memory medium [60] any of the received eye-tracking data, intermediate results at any stage(s) of the analysis, and/or test report [50].

Neurological disorders detected by system [100] can include reading function, such as a compromise in encoding and recognition of targets, a compromise in attentional processes, a compromise in cognitive resources, or any combination thereof. In other embodiments the disorders detected can include Multiple sclerosis (MS), Attention deficit-hyperactive disorder (ADHD), Parkinson disorder (PD), Alzheimer disease (AD), etc.

In some embodiments, processor [20] receives eye-tracking data from eye-tracker [10] while subject [5] views each of one or more targets [30]. Processor [20] measures gaze durations of subject [5] on each target [30] viewed by subject [5]. Processor [20] calculates an average gaze duration on each of the targets [30] by subject [5]. If an average of the gaze durations on targets [30] of subject [5] is longer than an average gaze duration for a control group, then processor [20] reports in test report [50] that a compromise in a target encoding and recognition process is detected in subject [5].

In some embodiments processor [20] additionally, or alternatively, counts a number of ocular fixations performed by subject [5] while viewing each of the targets [30]. If the number of ocular fixations performed by subject [5] while viewing the targets [30] is higher than for a control group, then processor [20] reports in test report [50] that a compromise in the attentional processes is detected in subject [5].

In some embodiments, processor [20] receives pupil diameter data from means [17] of measuring pupil diameter while subject [5] performs activities requiring lower cognitive effort. Processor [20] further receives pupil diameter data from means [17] of measuring pupil diameter while subject [5] performs activities requiring a stronger cognitive effort than for the activities requiring lower cognitive effort. If an average pupil diameter of subject 5 while performing the activities requiring the stronger cognitive effort does not show an increase over an average pupil diameter of subject [5] while performing the activities requiring lower cognitive effort, then processor [20] reports in test report [50] that a compromise in cognitive resources is detected in subject [5].

The control group may comprise a statistically representative cross-section in the same demographic sector as subject [5] (e.g., the same gender, race, national culture, age group, and/or other demographic features of subject [5]). Eye-tracking data for the control group may be obtained by system [100] or otherwise gathered from previous research studies and/or clinical studies. Where the average gaze duration or number of ocular fixations of subject [5] is within a selected margin about one standard deviation of a distribution of the corresponding figure for the control group of the average figure for the control group, system [100] may treat the average gaze duration or number of ocular fixations of subject [5] as equal to the average corresponding figure for the control group.

It is understood that eye tracking data received by processor [20] may be a series of eyeball positions measured by eye tracker [10], which processor [20] analyzes to find gaze durations and ocular fixations of subject [5]. Alternatively, processor [20] may receive a series of pre-processed signals from eye tracker [10], each signaling a gaze duration or that an ocular fixation has occurred. The signals may optionally be accompanied with metadata (e.g., eyeball position, time, and/or length of the ocular fixation).

### Multiple Sclerosis

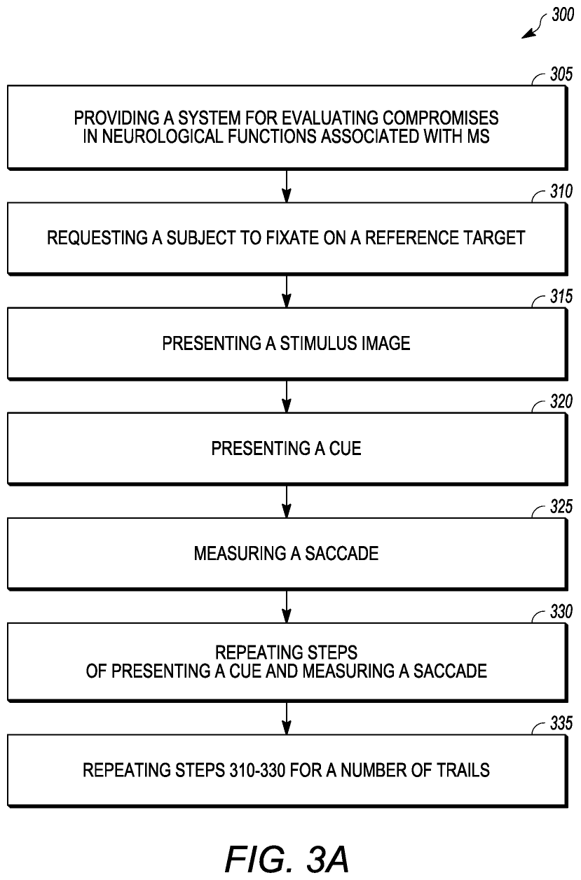

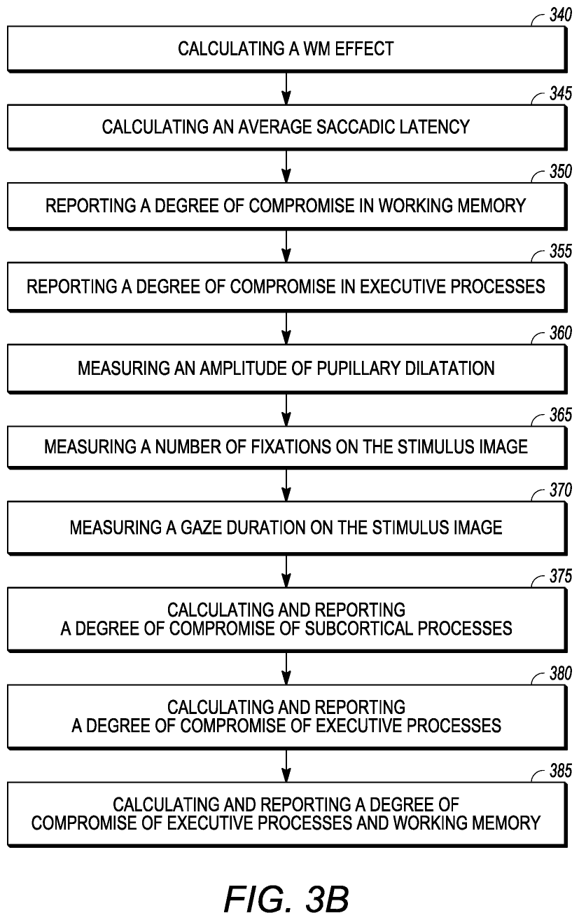

Reference is now made to FIGS. 3A and 3B, showing a method [300] for evaluating compromises in neurological functions associated with Multiple Sclerosis [MS], according to some embodiments of the invention. Method [300] comprises steps of:
a. providing a system for evaluating compromises in neurological functions associated with MS [305];
b. requesting a subject to fixate on a reference target of a chart [310];
c. for a number of repetitions, presenting a stimulus image in one of a plurality of zones on the chart to the subject [315]; the subject is requested to remember which zone each stimulus image appeared and in what order;
d. presenting to the subject a cue corresponding to one of the presented stimulus images [320];
e. measuring a saccade of the subject [325] in response to the step of presenting a cue; the subject is requested to look at the zone in which was the presented stimulus image corresponding to the cue;
f. repeating steps of presenting a cue and measuring a saccade [330];
g. repeating steps b-f for a number of trials [335];
h. calculating one or more of:
i. a WM effect [340] (i.e. WM effect is a measure that increases when WM demand increases. For each cue number, the WM effect is represented by the ratio between the number of errors reported by the subject through all the trials, and the number of trials); and
ii. an average saccadic latency [345], saccadic latency defined as an amount of time for the subject to initiate a saccade to the zone; and
i. reporting one or more of:
i. a degree of compromise in working memory [350], with increased WM effect; and
ii. a degree of compromise in executive processes [355], with increased saccadic latency; wherein the method further comprises additional steps, performed during the step of presenting a stimulus image [315]; during which the subject is further requested to look at the stimulus image;
j. the additional steps comprising measuring one or more of: i. an amplitude of pupillary dilatation of the subject [360];
ii. a number of fixations made by the subject on the stimulus image [365]; and
iii. a gaze duration by the subject on the stimulus image [370].
k. the additional steps further comprising calculating and reporting one or more of:
i. a degree of compromise of subcortical processes, with an unchanged amplitude on pupil dilatation [375];
ii. a degree of compromise of executive processes, with increased number of fixations [380]; and
iii. a degree of compromise of executive processes and working memory, with increased gaze duration [385].

The method employs an intelligent algorithm to analyze the subject, utilizing the following variables:
a. Total number of ocular fixations of a subject while performing the n-Back Task.
b. Identification Number of n-Back Task Trial (i.e. if there are 20 n-Back Tasks Trials, the 5th trial is identified with the number 5. The 20th trial is identified with the number 20 etc.)
c. Trial Part i.e., 1, 2 and 3.
d. Part of the Trial i.e., encoding; retrieval.
e. Pupil diameter of the subject while performing n-Back Task.
f. Number of blinks coming from the left eye, the right eye or from both eyes.
g. Microsaccades; Factors of Form (FF):
i. HEWI: shows the microsacade's height/width relationship. ii. AREA: shows the area of the rectangle in which the microsaccade is inscribed.
iii. LONG: is the longitude of the horizontal-vertical plane trajectory of the microsaccade.
iv. ANG: is the sum of all the angles in the plane horizontal-vertical plane of the microsaccade.
v. AANG: is the sum of all the absolute values of angles in radians in the plane horizontal-vertical plane of the microsaccade. These las two FF give an estimation of the microsaccadic trajectory regularity.
vi. MOD and THETA: are the modulus and the angle of the polar coordinates of the sum of the cartesian coordinates. They give a spatial orientation of the microsaccade relative to the median of the fixation. vii. TIME: is the time duration in milliseconds of the microsaccade.
viii. VMIN and VMAX: are the minimum and maximum velocities of the microsaccades in degrees per second.
ix. Microsaccade rate: is the instantaneous rate in each time bin.
x. Directional congruency: is the congruency between the microsaccade direction and the location of the stimulus.
h. Eye position coming from the left eye, the right eye or from both eyes (i.e., abscissa and ordinate coordinate) while performing the n-Back Task.
i. Saccade amplitude while processing the targets.
j. Saccade latency.
k. Fixation sequence (i.e., ocular behavior) while processing the targets. The sequence will be available from images, from matrices, etc.
l. Distance between the fixation point of the Right Eye and the Left Eye while performing the processing targets.
m. Filia information of the subject (i.e., age; years of education; sex; ethnic group; occupation; hours per week of physical activity).
n. Fixation duration while processing targets.
o. Gaze duration while processing targets.
p. Number of fixations on each target.
q. Number of fixations outside each target.

The measurements made while presenting the stimulus image (feature j in method[300]) provides information during encoding, which occurs while the subject identifies the location of the visual stimulus for the first time. In pilot studies made by inventors, subjects with MS were found to be impaired when encoding visual information (e.g., subjects made many fixations on the display). Measurements during encoding are in addition to the measurements taken during recognition, when presented with cues after the visual stimuli are presented as in the study of Fielding et al. (steps a-i in method [300]). Taken together, performance of the subject during both encoding and recognition can help identify additional deficiencies (namely, degrees of compromise of subcortical processes, executive processes, and/or executive processes) and provide greater insight into the condition of the subject than performance during recognition alone.

### Reading

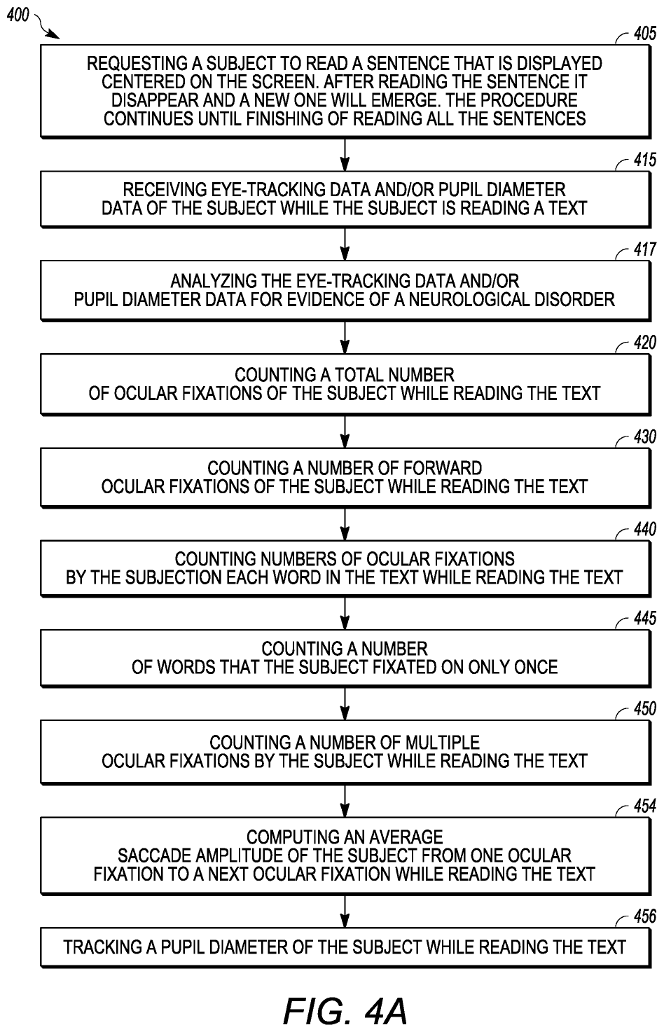

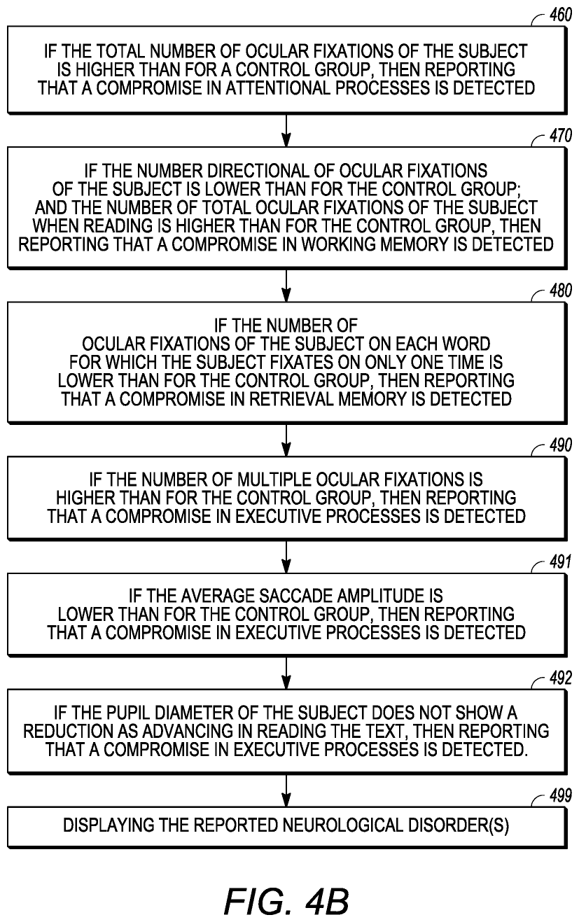

Reference is now made to FIGS. 4A and 4B, showing a method for measuring general cognitive performance and for detecting one or more neurological disorders of a subject, by measuring eye movements and/or pupil diameter of the subject while the subject is reading, according to some embodiments of the invention.

Method [400] comprises steps of providing a system for measuring general cognitive performance and for detecting the presence of one or more neurological disorders by measuring eye movements and/or pupil diameter; receiving eye-tracking data and/or pupil diameter data of a subject reading a text; analyzing the eye-tracking data for evidence of one or more neurological disorders; and displaying a report of detection of the neurological disorder(s).

In some embodiments, method [400] comprises steps of counting a total number of ocular fixations of the subject while the subject is reading the text [405]; and reporting that a compromise in attentional processes is detected, if the total number of ocular fixations of the subject when reading the text is higher than for a control group [460].

In some embodiments, method [400] further comprises steps of counting a total number of ocular fixations of the subject while reading the text [405]; counting a number of forward ocular fixations of the subject while reading the text [430]; and reporting that a compromise in working memory is detected, if the number of forward ocular fixations of the subject is higher than for the control group and the number of total ocular fixations of the subject when reading is higher than for the control group [470].

Physiologically, a compromise in working memory is correlated with deterioration in the frontal lobe. In some embodiments, reporting of a compromise in working memory [470] may be used in additional treatment. For example, if neurosurgery is indicated, method [400] may be followed by studying brain imagery of the subject's frontal lobe.

In some embodiments, method [400] comprises steps of counting numbers of ocular fixations by the subject on each word in the text while the subject is reading the text [440]; counting a number of words that the subject fixated on only once [445]; and reporting that a compromise in retrieval memory is detected, if the number of words that subject fixated on only once is lower than for the control group [480].

Physiologically, a compromise in retrieval memory is correlated with deterioration in the temporal lobe. In some embodiments, reporting of a compromise in retrieval memory [480] may be used in additional treatment. For example, if neurosurgery is indicated, method [400] may be followed by studying brain imagery of the subject's frontal lobe.

In some embodiments, method [400] comprises steps of counting a number of multiple ocular fixations of subject while reading the text [450]; and reporting that a compromise in executive processes is detected, if the number of multiple ocular fixations is higher than for the control group [490].

In some embodiments, method [400] comprises steps of computing an average saccade amplitude of the subject from one ocular fixation to a next ocular fixation while reading the text [454]; and reporting that a compromise in executive processes is detected, if the average saccade amplitude is lower than for the control group [491].

In some embodiments, method [400] comprises steps of tracking a pupil diameter of the subject while reading the text [456]; and reporting that a compromise in executive processes is detected, if the pupil diameter of the subject does not show a reduction as advancing in reading the text [492].

Physiologically, a compromise in executive processes is correlated with deterioration in the frontal, temporal, and/or parietal lobes. In some embodiments, reporting of a compromise in executive processes [490-491-492] may be used in additional treatment. For example, if neurosurgery is indicated, method [400] may be followed by studying brain imagery of the subject's frontal, temporal, and/or parietal lobes.

The system and method [400] were tested on 50 Healthy Controls and 50 Mild AD Patients. Both groups read 40 regular sentences.

TABLE 1

| Test | Control Group | AD Group |
|---|---|---|
| Attentional Processes | 520 (21) | 882 (317) |
| Executive Processes | 14 (8) | 37 (6) |
| Working Memory | 85 (14) | 61 (9) |
| Retrieval Memory | 30 (6) | 12 (11) |

### BIBLIOGRAPHY

The above rules are based in part upon findings in the following studies:
1. Fernández G, Mandolesi P, Rotstein N P, Colombo O, Agamennoni O, Politi L E. (2013) Eye movement alterations during reading inpatients with early Alzheimer disease. Invest Ophthalmol Vis Sci. pii: iovs.13-12877v1. doi: 10.1167/iovs.13-12877.
2. Fernández G., Manes F., Politi L., Orozco D., Schumacher M., Castro L., Agamennoni O., Rotstein N. (2016). Patients with Mild Alzheimer Disease Fail When Using Their Working Memory: Evidence from the Eye Tracking Technique. Journal of Alzheimer Disease; 50, 827-828.
3. Fernández, G., Laubrock, J., Mandolesi P., Colombo O., Agamennoni O. (2014) Registering eye movements during reading in Alzheimer disease: difficulties in predicting upcoming words. Journal of Clinical and Experimental Neuropsychology; 36, 302-16.
4. Fernández G., Sapognikoff M., Guinjoan S., Orozco D., Agamennoni O. (2016). Word processing during reading sentences in patients with schizophrenia: evidences from the eye tracking technique. COMPREHENSIVE PSYCHIATRY; 68, 193-200.
5. Fernández G, Manes F, Rotstein N, Colombo O, Mandolesi P, Politi L, Agamennoni O. (2014) Lack of contextual-word predictability during reading inpatients with mild Alzheimer disease. Neuropsychologia; 62, 143-51.
6. Fernández G., Schumacher M., Castro L., Orozco D., Agamennoni O., (2015). Patients with Alzheimer disease produced shorter outgoing saccades when reading sentences. Psychiatry Research, 229, 470-478.
7. Fernández G., Biondi J., Castro S., Agamennoni O. (2017). Pupil size behavior during online processing of sentences. Journal of Integrative Neurosciences 15(4) 485-496

### Memory Binding

Non-limiting embodiments of the invention are now described in detail.

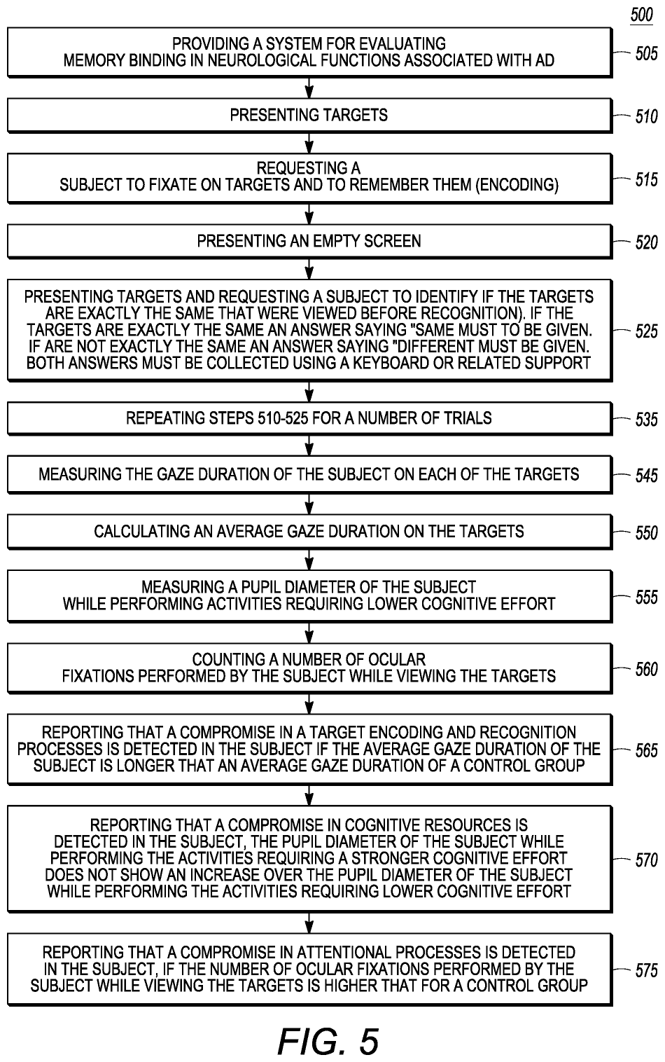

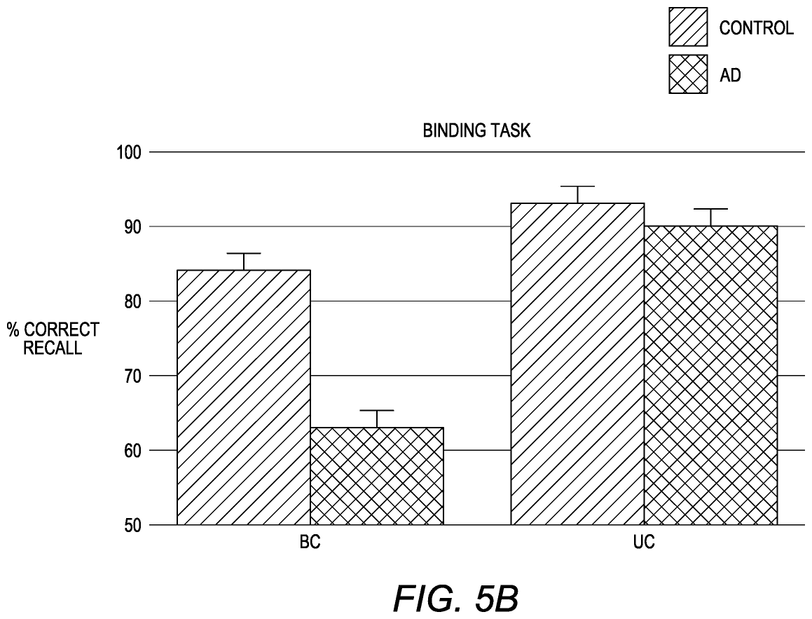

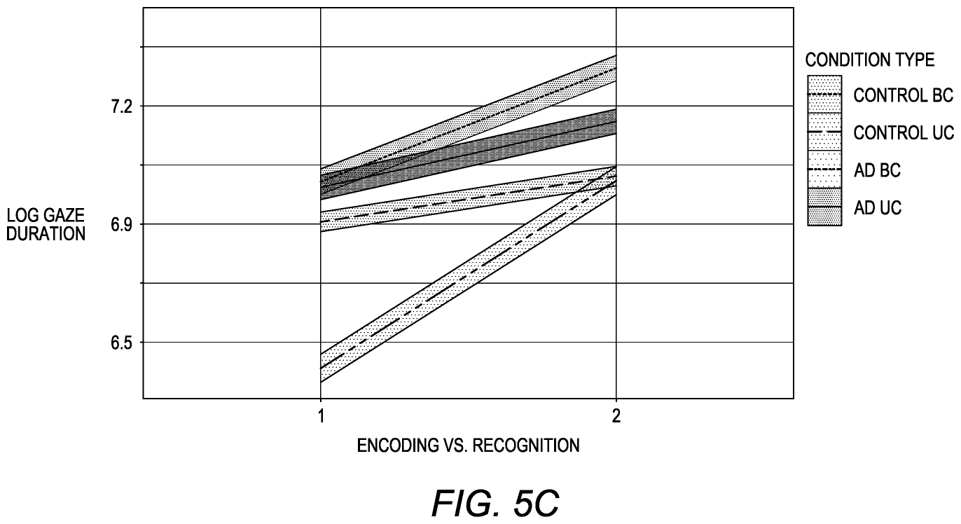

Reference is now made to FIG. 5, showing a method [500] for detecting a disorder of memory binding function in a subject, according to some embodiments of the invention.

Method comprises a step [505] of providing a system for detecting a disorder of memory binding function in a subject.

In some embodiments, method [500] comprises a step [510-535] of viewing by a subject of one or more targets; a step [545] of measuring a gaze duration of the subject on each of said targets; a step [550] of calculating an average gaze duration of the targets by the subject; and a step [565] of reporting that a compromise in a target encoding and recognition process is detected in the subject, if an average of the gaze durations of the subject is longer than an average gaze duration for a control group.

In some embodiments, method [500] comprises a step [555] of measuring one or more pupil diameters of the subject while performing activities requiring lower cognitive effort (e.g., recognizing three targets or distinguishing between targets; and a step [570] of reporting that a compromise in cognitive resources is detected in subject [5], if an average pupil diameter of subject [5] while performing the activities requiring a stronger cognitive effort does not show an increase over an average pupil diameter of subject [5] while performing activities requiring lower cognitive effort.

In some embodiments, method [500] comprises a step [560] of counting a number of ocular fixations by subject [5] while viewing the targets [30]; and a step [575] of reporting that a compromise in attentional processes is detected in subject [5], if the number of ocular fixations performed by subject [5] while viewing the targets [30] is higher than for the control group.

### BIBLIOGRAPHY

The above rules are based in part upon findings in the following studies:
1. Fernández G, Mandolesi P, Rotstein N P, Colombo O, Agamennoni O, Politi L E. (2013) Eye movement alterations during reading inpatients with early Alzheimer disease. Invest Ophthalmol Vis Sci. pii: iovs.13-12877v1. doi: 10.1167/iovs.13-12877.
2. Fernández G., Manes F., Politi L., Orozco D., Schumacher M., Castro L., Agamennoni O., Rotstein N. (2016). Patients with Mild Alzheimer Disease Fail When Using Their Working Memory: Evidence from the Eye Tracking Technique. Journal of Alzheimer Disease; 50, 827-828.
3. Fernández, G., Laubrock, J., Mandolesi P., Colombo O., Agamennoni O. (2014) Registering eye movements during reading in Alzheimer disease: difficulties in predicting upcoming words. Journal of Clinical and Experimental Neuropsychology; 36, 302-16.
4. Fernández G., Sapognikoff M., Guinjoan S., Orozco D., Agamennoni O. (2016). Word processing during reading sentences in patients with schizophrenia: evidences from the eye tracking technique. COMPREHENSIVE PSYCHIATRY; 68, 193-200.
5. Fernández G, Manes F, Rotstein N, Colombo O, Mandolesi P, Politi L, Agamennoni O. (2014) Lack of contextual-word predictability during reading inpatients with mil dAlzheimer disease. Neuropsychologia; 62, 143-51.
6. Fernández G., Schumacher M., Castro L., Orozco D., Agamennoni O., (2015). Patients with Alzheimer disease produced shorter outgoing saccades when reading sentences. Psychiatry Research, 229, 470-478.
7. Fernández G., Biondi J., Castro S., Agamennoni O. (2017). Pupil size behavior during online processing of sentences. Journal of Integrative Neurosciences 15(4) 485-496.
8. Biondi J., Fernandez G., Castro S., Agamennoni O. (2018). Eye-movement behavior identification for Alzheimer Disease diagnosis. Journal of Integrative Neurosciences (in Press).
9. Fernández, Orozco, Agamennoni, Schumacher, Sañudo, Biondi, Parra. (2018). Visual Processing during Short-Term Memory Binding in Mild Alzheimer's Disease. J Alzheimers Dis.; 63(1):185-194. doi: 10.3233/JAD-170728.

### Parkinson Disease (PD) and Attentional Deficit Hyperactive Disorders (ADHD)

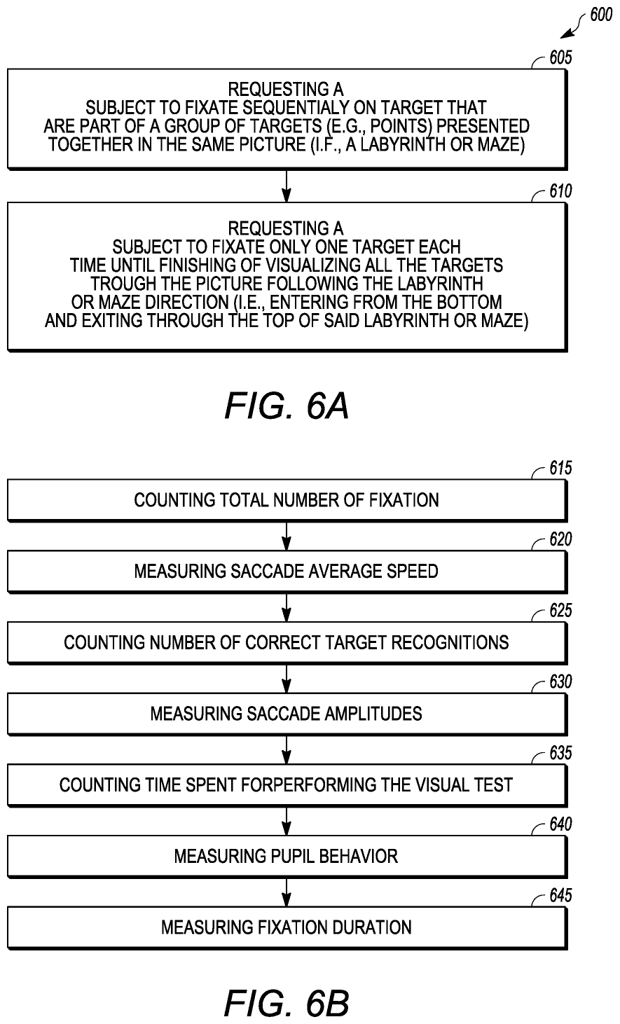

Reference is now made to FIGS. 6A and 6B showing a method for detecting one or more cognitive, neurological and behavioral impairments of a person, by measuring eye movements and/or pupil diameter of the person while the person is performing the visual test, according to some embodiments of the invention.

Method [600] comprises steps of providing a system for detecting the presence of one or more cognitive impairments and neurological disorders by measuring eye movements while a person is visualizing, recognizing, maintaining, controlling, inhibiting and sequencing targets; receiving eye-tracking data of a person visualizing, recognizing, maintaining, controlling, inhibiting and sequencing targets; analyzing the eye-tracking data for evidence of one or more cognitive impairments and neurological disorders; and displaying a report of detection of the cognitive impairments and neurological disorder(s).

In some embodiments, method [600] comprises steps of counting a total number of ocular fixations [615] of the person while the person is performing the visual test; and reporting that a compromise in attentional, executive and inhibitory processes is detected, if the number of ocular fixations of the person is higher than for a control group. [0100] In some embodiments, method [600] comprises steps for calculating the saccade average speed [620] of the subject [5] from one target to the other one, while the subject [5] is performing the visual test; reporting that a compromise in executive functions is detected, if the saccade average speed that person did is lower than for the control group. [0101] Physiologically, a slower saccade speed is correlated with deterioration in frontal eye fields, basal ganglia and superior colliculus. In some embodiments, reporting of a compromise in saccade speed may be used in additional treatment.

In some embodiments, method [600] comprises steps of counting a number of correct target recognitions of person while performing the visual test [625]; and reporting that a compromise in working memory is detected, if the number of correct target recognitions is lower than for the control group.

Physiologically, a compromise in working memory is correlated with a deterioration in Prefrontal Cortex and in the Posterior Parietal Cortex. In some embodiments, reporting of a compromise in working memory, inhibition processes and mental flexibility may be used in additional treatment.

In some embodiments, method [600] comprises steps of computing an average saccade amplitude from one ocular fixation to a next ocular fixation [630]; and reporting that a compromise in executive processes is detected, if the average saccade amplitude is lower than for the control group.

In some embodiments, method [600] comprises steps of tracking a pupil diameter of the person while performing the visual test [640]; and reporting that a compromise in attentional processes is detected, if the pupil diameter of the subject does not show an increase as advancing in performing the visual test.

Physiologically, a compromise in attentional processes is correlated with deterioration in the locus coeruleus, the noradrenergic system and in the superior colliculus. In some embodiments, reporting of a compromise in the executive processes may be used in additional treatment.

In some embodiments, method [600] comprises steps of computing the total time spent by the person while performing the visual trial [635]; and reporting that a compromise in attentional processes is detected, if the total time needed for performing the trial is major that the reported for the control group.

Physiologically, a compromise in attentional and inhibitory processes and in mental flexibility is correlated with deterioration in the prefrontal cortex, the posterior parietal cortex, the prefrontal striatal cerebellar and prefrontal striatal thalamic circuits. In some embodiments, reporting of a compromise in executive processes may be used in additional treatment.

In some embodiments, method [600] comprises steps of calculating fixation durations on targets of person while performing the visual test [645]; and reporting that a compromise in working memory is detected, if the fixation duration on targets is lower than for the control group.

Physiologically, a compromise in attentional and inhibitory processes and in mental flexibility is correlated with deterioration in the prefrontal cortex, the frontal eye fields and in the dorso-parietal cortex. In some embodiments, reporting of a compromise in executive processes may be used in additional treatment.

The method employs an intelligent algorithm to analyze the subject, utilizing the following variables:
a. Total number of ocular fixations of a subject while performing the Visual Test.
b. Identification Number of each target depending of its place in the labyrinth or maze.
c. Pupil diameter of the subject while performing the visual Test.
d. Number of blinks coming from the left eye, the right eye or from both eyes.
e. Microsaccades; Factors of Form (FF):
i. HEWI shows the microsacade's height/width relationship.
ii. AREA: shows the area of the rectangle in which the microsaccade is inscribed.
iii. LONG: is the longitude of the horizontal-vertical plane trajectory of the microsaccade.
iv. ANG: is the sum of all the angles in the plane horizontal-vertical plane of the microsaccade.
v. AANG: is the sum of all the absolute values of angles in radians in the plane horizontal-vertical plane of the microsaccade. These las two FF give an estimation of the microsaccadic trajectory regularity.
vi. MOD and THETA: are the modulus and the angle of the polar coordinates of the sum of the cartesian coordinates. They give a spatial orientation of the microsaccade relative to the median of the fixation.
vii. TIME: is the time duration in milliseconds of the microsaccade.
viii. VMIN and VMAX: are the minimum and maximum velocities of the microsaccades in degrees per second.
ix. Microsaccade rate: is the instantaneous rate in each time bin.
x. Directional congruency: is the congruency between the microsaccade direction and the location of the stimulus.
f. Eye position coming from the left eye, the right eye or from both eyes (i.e., abscissa and ordinate coordinate) while performing the visual Task.
g. Saccade amplitude while processing the targets.
h. Saccade latency.
i. Fixation sequence (i.e., ocular behavior) while processing the targets. The sequence will be available from images, from matrices, etc.
j. Distance between the fixation point of the Right Eye and the Left Eye while performing the processing targets.
k. Filia information of the subject (i.e., age; years of education; sex; ethnic group; occupation; hours per week of physical activity).
l. Fixation duration while processing targets.
m. Number of fixations on each target.
n. Number of fixations outside each target.
o. Total visual Task time (i.e., how much time spent the subject for performing the entire trial).

This method [600] was tested on subjects with PD and ADHD and compared to healthy controls:

TABLE 2
Parkinson’s Disease

| | CONTROL | | PM | |
|---|---|---|---|---|
| Mean GAZING (MS) | 283 | (±42.4) | 359.2 | (±29.5) |
| % Correct Fixation | 95% | (±3) | 81% | (±6) - |

ADHD

| | CONTROL | | ADHD | |
|---|---|---|---|---|
| Mean GAZING (MS) | 283 | (±42.4) | 370.3 | (±33.1) |
| % Correct Fixation | 95% | (±3) | 73% | (±7) |

### Evaluation of Treatment Regimens

Described herein are methods that use the systems and techniques described above to evaluate the treatment regimen (e.g., medicaments such as drugs, medicines etc.) being followed by the patient in accordance with a medical practitioner's instructions. In this way the medical practitioner and/or a pharmaceutical manufacturer can better track the effectiveness of the treatment regimen on the patient and alter or supplement the regimen as necessary based on that evaluation throughout the course of the disease. For example, drugs or other medicaments that may be evaluated include neurological and/or psychiatric drugs that have a neurological and/or psychiatric effect.

For purposes of illustration only and not as a limitation on the methods described herein, examples will be presented below in which eye movements are modeled in MS patients who receive different drugs (e.g., Dimethyl fumarate, Fingolimod, Cladribine, Ofatumumab) or treatments that (a) decrease inflammation and prevent nerve damage that can cause symptoms of multiple sclerosis); (b) test Sphingosine-l-phosphate receptor modulator, which sequesters lymphocytes in the lymphocytes nodes, preventing them from contributing to an autoimmune reaction); (c) check an Inmune suppressor agent that works on the lymphocyte's pathway) and (d) analyze the effect of Monoclonal Antibodies for inhibiting the activation of lymphocyte B. In addition, we explain how medical practitioners, pharmaceutical manufacturers and others can evaluate the effects of these medicaments and any other treatments on cognitive performance and high-level motor abilities.

Understanding various medicaments' (e.g., drugs, etc) impact on the Central Nervous System (CNS) and on the peripheral nervous system (PNS) through the analysis of eye movements when performing well-defined activities as those reported for us (e.g., go no-go and n-back test) would allow medical practitioners to test at what level and with what efficacy a medicament or treatment are producing the expected impact on the patient's Disease course. In this sense, medical practitioners will have access to a novel tool for testing medicaments effects on patient's cognitive and fine motor alterations. In addition, pharmaceutical companies will have also an objective and quantifiable measurement about their medicaments' impact on well-defined domains, opening a new path for analyzing who should repeat a new administration of the drug (including the doses) and also what are the patients that better assimilate their medicaments, among other things.

Some embodiments of the methods described herein may perform one or more of the following: calculating, modelling and reporting one or more effects of drugs (e.g., Dimethyl fumarate, Fingolimod, Cladribine, Ofatumumab, Interferon-Beta) or treatments in order to test if there is (a) a decrease on the inflammation and nerve damage that can cause symptoms of multiple sclerosis; (b) a damage on the receptor of the Sphingosine-l-phosphate modulator, which sequesters lymphocytes in the lymphocytes nodes, preventing them from contributing to an autoimmune reaction; (c) a damage on the Inmune suppressor agent that works on the lymphocyte's pathway and/or (d) a therapeutic effect of Monoclonal Antibodies for inhibiting the activation of lymphocyte B on some well-defined neurological processes and related cognitive activities.

We apply mathematical models where the considered dependent variable could be, for example, saccade amplitude, fixation duration, pupil behavior; and predictors could be motor scales, cognitive scales, years of diagnosis of the disease and treatments (i.e., drugs), among others. We obtain regression coefficients, standard errors and t-values from each model in order to understand what the impact of a treatment is on a particular eye movement (e.g., saccade amplitude), on a combined set of eye movements, on related cognitive functions and on related areas of the brain. We take a first measurement (Baseline) and repeat the exercise (when required) in order to check if the treatment is working properly.

The following examples explains how to use saccade amplitude as a dependent variable: The saccade amplitude depends on the strategy developed by the person evaluated to scan figures while performing a particular test. If the test is the n-back task, because of the nature of the test, a person performing better will do longer saccades. Longer saccades suggest that working memory is performing well, while shorted saccades imply a poor performance (as shown previously in this patent). For the saccade amplitude to be longer, in this case, the dorsolateral prefrontal cortex, basal ganglia and superior colliculus must be preserved. The reason behind this statement is that the dorsolateral prefrontal cortex, the basal ganglia, and the superior colliculus are key in defining where the different fixations will take place (hence, impacting on the saccade amplitude) (Fielding et al., 2015). In addition, saccades should be longer when the performance in cognitive (e.g., The Symbol Digit Modalities Test) and motor (e.g., The Expanded Disability Status Scale) scales show better outputs. The reason behind this is that better cognitive scales outcome positively correlate with more preserved Working Memory, while better motor skills positively correlate with more preserved high-level motor functions. For this reason, the Symbol Digit Modalities Test and the Expanded Disability Status Scale can be used as predictors.

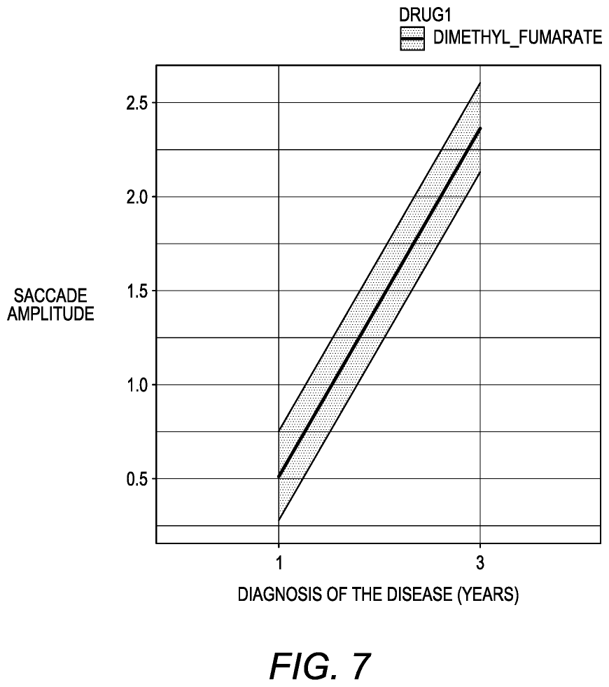

If a person is been treated with Dimethyl fumarate (which could (a) produce a decrease on the inflammation and nerve damage that can cause symptoms of multiple sclerosis) and the saccade amplitude while conducting the N-Back task is longer, it can be inferred that the treatment has a positive impact on Working Memory and on the dorsolateral prefrontal cortex, basal ganglia and superior colliculus. In FIG. 7, a positive impact can be seen in a patient taking Dimethyl Fumarate, showing longer saccade amplitude as treatment progresses (4 years of treatment).

The following examples explains how to use pupil behavior as a dependent variable: A patient's pupil behavior varies depending on the cognitive effort performed by the patient in a particular moment. The size of the pupil increases when a task is more demanding (as explained previously in this patent). When performing the N-Back Task, given the complexity of the test, the pupil size must increase. This particular behavior suggests that the noradrenergic system and also the locus coeruleus are responding properly as the cognitive load increase. This statement is pupil size and cognitive load (Fernández et al, 2021). If a person is being treated with Interferon-Beta (which could (b) reduces damage on the Inmune suppressor agent that works on the lymphocyte's pathway) and the pupil size increases as the cognitive load increases, it can be inferred that the treatment has a positive impact on the amount of Working Memory resources used (Sweller et al., 2011) and in the noradrenergic system and locus coeruleus.

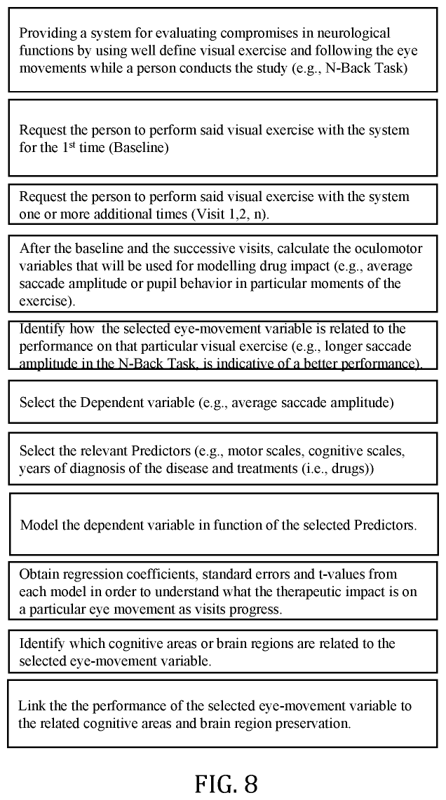

FIG. 8 is a flowchart showing a method for identifying specific alterations in subjects with defined disease analyzing oculomotor patterns when using specific visual stimuli, where a specific drug or treatment would enhance visual processing, cognitive performance and related brain activities.

### Additional Details and Examples of Methods and Systems for Evaluating Treatment Regimens

### N-Back Task

In one example, in order to check specific medicament (e.g, drug, medicine, etc.) or treatment impact, a method is presented to evaluate compromises in neurological disorders, fine-motor skills, executive processes, decision making, processing speed and cognitive capabilities associated with Multiple Sclerosis [MS], the method comprising
a. providing a system for evaluating compromises in neurological disorders, fine-motor skills, executive processes, decision making, fine motor skills and cognitive capabilities associated with MS;
b. requesting a subject to fixate on a reference target of a chart, where the chart includes multiple regions (e.g., rectangles) placed in different zones;
c. for a number of repetitions, presenting a stimulus image in one of the zones to the subject, the subject being requested to remember which zone each stimulus image appeared and in what order;
d. presenting to the subject the chart without including the stimulus image presented in step c, where the subject is requested to fixate in a zone that is where the stimulus image of step c appeared;
e. measuring a saccade of the subject in response to the presenting of step d who is requested to look at the zone in which was presented the stimulus image presented in step c;
f. repeating steps d and e of presenting a chart and measuring a saccade;
g. repeating steps b-f for a number of trials modifying a time in which the stimulus images are shown;
h. calculating one or more of
i. a WM effect, wherein the WM effect is a measure that increases when WM demand increases. For each stimulus image fixated, the WM effect is represented by the ratio between the number of errors reported by the subject through all the trials, and a number of trials); and
ii. an average saccadic latency, saccadic latency defined as an amount of time for the subject to initiate a saccade to the zone; and reporting one or more of
iii. a degree of compromise in working memory, with increased the WM effect; and
i. a degree of compromise in executive processes, with increased saccadic latency;
j. wherein the method further comprises additional steps comprising measurements performed during the step of presenting a stimulus image, during which the subject is further requested to look at the stimulus image; the measurements comprising measuring one or more of
a. an amplitude of pupillary dilatation of the subject;
b. a number of fixations made by the subject on the stimulus image;
c. a gaze duration by the subject on the stimulus image;
d. binocular disparity by the while visual exploring and target visualization;
e. target hit by the subject fixate where the visual stimulus was present previously;
f. number of consecutive target hits by the subject when considering a trial;
g. Number of blinks coming from the left eye, the right eye or from both eyes;
h. an intelligent algorithm with ocolumotor behaviour as income for classifying person's performance;
i. Microsaccades' Factors of Form (FF):
i) HEWI: shows the microsacade's height/width relationship. ii) AREA: shows the area of the rectangle in which the microsaccade is inscribed;
ii) LONG: is the longitude of the horizontal-vertical plane trajectory of the microsaccade.
iii) ANG: is the sum of all the angles in the plane horizontal-vertical plane of the microsaccade;
iv) AANG: is the sum of all the absolute values of angles in radians in the plane horizontal-vertical plane of the microsacaccade. These las two FF give an estimation of the microsaccadic trajectory regularity;
v) MOD and THETA: are the modulus and the angle of the polar coordinates of the sum of the cartesian coordinates. They give a spatial orientation of the microsaccade relative to the median of the fixation;
vi) TIME: is the time duration in milliseconds of the microsaccade.
vii) VMIN and VMAX: are the minimum and maximum velocities of the microsaccades in degrees per second;
viii) Microsaccade rate: is the instantaneous rate in each time bin;
ix) Directional congruency: is the congruency between the microsaccade direction and the location of the stimulus;
k. Obtaining eye position information coming from the left eye, the right eye or from both eyes (i.e., abscissa and ordinate coordinate) while performing visual exploration.

It should be noted that any one or more (or all) of items calculated in step h may be omitted. Likewise, any one or more (or all) of the additional steps set forth in step i may be omitted.

Moreover, in some embodiments the additional steps may further comprise calculating, modelling and reporting one or more effects of drugs (e.g., Dimethyl fumarate, Fingolimod, Cladribine, Ofatumumab, Interferon-Beta) or treatments that (a) decrease inflammation and prevent nerve damage that can cause symptoms of multiple sclerosis); (b) test the Sphingosine-l-phosphate receptor modulator, which sequesters lymphocytes in the lymphocytes nodes, preventing them from contributing to an autoimmune reaction); (c) check an inmune suppressor agent that works on the lymphocyte's pathway) and/or (d) analyze the effect of Monoclonal Antibodies for inhibiting the activation of lymphocyte B.

### GO NO-GO Task

In another example, a method (Go No-Go) and system is provided for evaluating compromises in neurological disorders, fine-motor skills, processing speed, decision making and cognitive processes associated with Multiple Sclerosis.

In one particular example, a system and method is provided for detecting one or more neurological disorders and/or measuring, fine-motor skills, processing speed, decision making, and cognitive processes in a subject by measuring eye movements, oculomotor features or pupil behaviour, the measuring of eye movements being performed while the subject is visualizing (i.e., to form a picture of something in the mind, in order to imagine or remember it), recognizing (i.e., to identify something from having encountered it before), maintaining (i.e., to keep in an existing memory), controlling (i.e., to exercise restraint or direction over), inhibiting (i.e., to prevent or hold back from doing something), fixating (i.e., to focus the eyes on something) and analyzing targets. The system may comprise:
a. an eye tracker, configured to monitor eye movements of a subject while the subject is visualizing, recognizing, maintaining, controlling, fixating and analyzing targets;
b. a processor configured to receive data from the eye tracker while the subject is visualizing, recognizing, maintaining, controlling, fixating and analyzing the targets; and
c. a display configured to display a test report received from the processor, wherein the processor is further configured to analyze the eye-tracking data for evidence of one or more neurological disorders or general cognitive performance and to report, in the test report, a detection of the one or more neurological disorders or a measure of cognitive performance of the subject.

In one particular implementation, the processor is further configured, upon receiving the eye-tracking data from the eye tracker, to perform one or more (or all) of the following:
a. count a total number of ocular fixations of a subject while visualizing, recognizing, maintaining, controlling, fixating and analyzing targets; and
b. if the total number of ocular fixations of a subject when visualizing, recognizing, maintaining, controlling, fixating and analyzing targets is higher than for a control group, then report in the test report that a compromise in attentional processes is detected;
c. count a number of correct landing positions of the subject while visualizing, recognizing, maintaining, controlling, fixating and analyzing the targets; and
d. if the number of correct landing positions of the subject is lower than for the control group; then report in the test report that a compromise in executive processes is detected;
e. count a number of right cue directed outgoing saccades while trying of visualizing, recognizing, maintaining, controlling, fixating, following and analyzing targets; and
f. if the percentage number of right cue (e.g., the direction of an arrow) directed outgoing saccades that the subject do is lower than for the control group, then report in the test report that a compromise in executive processes is detected;
g. count a number of opposite cue directions of outgoing saccades of the subject while trying of visualizing, recognizing, maintaining, controlling, fixating, following and analyzing targets;
h. if the percentage number of opposite cue directions of outgoing saccades is higher than for the control group, then report in the test report that a compromise in inhibitory processes is detected;
i. compute an average saccade amplitude from one ocular fixation to a next ocular fixation while visualizing, recognizing, maintaining, controlling, fixating, following and analyzing targets;
j. if the average saccade amplitude is lower than for the control group, then report in the test report that a compromise in executive processes is detected;
k. count saccade latency length of the subject while directing sending eyes for visualizing, recognizing, maintaining, controlling, inhibiting, fixating, following and analyzing targets;
l. if the saccade latency length (time) is higher than for the control group, then report in the test report that a compromise in speed processing is detected.
m. track the pupil diameter of the subject when visualizing, recognizing, maintaining, controlling, inhibiting, fixating, following and analyzing targets; and
n. if the pupil diameter of the subject does not show a modulation as advancing in visualizing, recognizing, maintaining, controlling, inhibiting, fixating, following and analyzing targets, then report in the test report that that a compromise in noradrenergic system is detected.
o. consider length of fixation duration of the subject while trying of visualizing, recognizing, maintaining, controlling, fixating, following and analyzing targets; and
p. if the length of fixation duration is longer than for the control group, then report in the test report that a compromise in on-line processing is detected;
q. consider gaze duration of the subject while trying of visualizing, recognizing, maintaining, controlling, fixating, following and analyzing targets;
r. if the length of gaze duration is longer than for the control group, then report in the test report that a compromise in on-line processing is detected;
s. count number of correct target recognized while visualizing, recognizing, maintaining, controlling, inhibiting, fixating, following and analyzing targets; and
t. if the number of correct target recognized is lower than for the control group, then report the in the test report that compromises in executive and working memory processes are detected;
u. count blinks coming from the left eye, the right eye or from both eyes when visualizing, recognizing, maintaining, controlling, inhibiting, fixating, following and analyzing targets;
v. apply an Intelligent algorithm with ocolumotor behaviour as income for classifying person's performance.
v. measure microsaccades' Factors of Form (FF):
i. HEWI: shows the micro-saccade's height/width relationship;
ii. AREA: shows the area of the rectangle in which the micro-saccade is inscribed;
iii. LONG: is the longitude of the horizontal-vertical plane trajectory of the micro-saccade;
iv. ANG: is the sum of all the angles in the plane horizontal-vertical plane of the micro-saccade;
v. AANG: is the sum of all the absolute values of angles in radians in the plane horizontal-vertical plane of the micro-saccade;
vi. FF gives an estimation of the micro-saccadic trajectory regularity;
vii. MOD and THETA: are the modulus and the angle of the polar coordinates of the sum of the cartesian coordinates. They give a spatial orientation of the micro-saccade relative to the median of the fixation;
viii. TIME: is the time duration in milliseconds of the micro-saccade;
ix. VMIN and VMAX: are the minimum and maximum velocities of the microsaccades in degrees per second;
x. Micro-saccade rate: is the instantaneous rate in each time bin;
xi. Directional congruency: is the congruency between the micro-saccade directionant the location of the stimulus;
w. Measure eye position coming from the left eye, the right eye or from both eyes (i.e., abscissa and ordinate coordinate) during visualizing, recognizing, maintaining, controlling, sequencing and analyzing targets;
x. measure total visualizing, recognizing, maintaining, controlling, fixating, following and analyzing targets time (i.e., the time that the subject spent when visualizing targets through a trial);
y. count a number of correct target recognized while visualizing, recognizing, maintaining, controlling, inhibiting, fixating, following and analyzing targets.

The processor may be further configured to perform additional steps that include calculating, modelling and reporting one or more effects of drugs (e.g., Dimethylfumarate, Fingolimod, Cladribine, Ofatumumab, Interferon-Beta) or treatments that (a) decrease inflammation and prevent nerve damage that can cause symptoms of multiple sclerosis);
(b) test Sphingosine-l-phosphate receptor modulator, which sequesters lymphocytes in the lymphocytes nodes, preventing them from contributing to an autoimmune reaction); (c) check an immune suppressor agent that works on the lymphocyte's pathway); and/or (d) analyze the effect of Monoclonal Antibodies for inhibiting the activation of lymphocyte B.

### Evaluation of Performance, Motor Skills and Cognitive Capabilities Using a Virtual Reality Environment

Described herein are systems and methods for combining virtual reality (VR), eye-tracking (ET), and motion sensors on limbs such as hands and feet to evaluate the changes of cognitive and motor abilities in both healthy and non-healthy persons using well-defined exercises. The application of VR and ET in cognitive exercises with motion sensors can improve the efficacy of the intervention and the ability to quantify cognitive and motor capabilities, enhancing the effectiveness of the training on a person. For example, the combination of VR, visual scanning and arm and leg movements, can provide new information about a person's decision-making processes and the integrity of brain circuits (e.g., what a person does when visualizing a shape and deciding to move the hand to touch something in a VR environment). Such a methodology would enhance a healthcare professional's ability to analyze, quantify and train cognitive capabilities and fine-motor skills.

The eye tracking system described herein may be incorporated in a conventional Head Mounted Display (TIMID), where a VR world is rendered and seen by the user. Each manufacturer of commercially available HMDs/VR devices performs eye tracking in a somewhat different way. However, the interface provided to developers allows them to access the vector (three numerical values) which indicates the direction that the eye is looking in the 3D virtual space created by the VR application. In this way integration between the eye-tracking system and the VR application is seamless. This interface is usually available in multiple languages/gaming development environments. Examples of some types of commercial VR devices that may employed include: HTC Vive Eye Pro; HP G2 Reverb Omnicept Edition; Varjo Aero and Fove 0. The controllers that are provided with the VR device generally will work to track hand motions. Some of them (like the Valve controllers) are compatible with bases which are independent of the VR Headset used. Virtual reality (VR) controllers play a pivotal role in unlocking captivating immersive encounters within virtual environments. These tools empower users to engage with and manipulate the digital realm, culminating in an exceptionally absorbing and involved experience. Fundamentally, a VR input device serves as a conduit for transmitting hand motion data to a computer system. This information is subsequently processed and harnessed to control objects existing within the simulated world. Currently, there are two primary categories of input devices in use: motion controllers and game controllers. Motion controllers employ accelerometers and gyroscopes to detect motion and orientation changes. They can also incorporate buttons, analog sticks, and various input mechanisms depending on the specific device. Motion controllers excel in scenarios where direct interaction with the virtual surroundings is paramount, such as exploratory first-person experiences. On the other hand, game controllers are more commonly associated with traditional gaming encounters. These controllers usually offer an array of input options, encompassing dual analog sticks and a multitude of buttons. In the most recent iteration of input devices, an added layer of immersion is achieved through the inclusion of haptic feedback. This augmentation enhances the virtual experience by providing tangible responses when interacting with elements within the virtual domain.

We apply mathematical models where the dependent variable that is considered could be, for example, saccade amplitude, fixation duration, pupil behavior, hand reaction time, tracking accuracy; and independent variables could be motor measurements, cognitive measurements, years of diagnosis of the disease, treatments, among others. We obtain regression coefficients, standard errors and t-values from each model in order to understand what the impact of a disease or a treatment or a training is on a particular eye movement (e.g., saccade amplitude) or limb movements (e.g., hand reaction time), on a combined set of eye movements, on related cognitive functions, on fine-motor pathways and on related areas of the brain.

The following illustrative examples explain how to use saccade amplitude as a dependent variable:

The saccade amplitude depends on the strategy developed by the person being evaluated to send the eyes to a particular shape while performing a particular test. If the test is the Go No-Go 3D, because of the nature of the test, a person performing better will perform longer saccades. Longer saccades suggest that working memory is performing well, while shorter saccades imply a poor performance (as previously discussed herein). For the saccade amplitude to be longer, in this case, the dorsolateral prefrontal cortex, basal ganglia and superior colliculus must be preserved. The reason behind this statement is that the dorsolateral prefrontal cortex, the basal ganglia, and the superior colliculus are key in defining where the different fixations will take place (hence, impacting on the saccade amplitude) (see Fielding, J. et al. Nat. Rev. Neurol. 11, 637-645 (2015); doi:10.1038/nrneurol.2015.174). For example, if a person diagnosed with Multiple Sclerosis performs the study, his/her saccades will be shorter and less accurate as the disabilities increase, and it can be inferred that the disease has a significant impact on working memory and on the dorsolateral prefrontal cortex, basal ganglia and superior colliculus.

The following examples explain how to use person's Hand Reaction as a dependent variable.

A person's Hand Reaction Time assesses the average time it takes for the person to initiate a manual response after visually perceiving a target. It may reflect the person's motor response time and coordination. This measurement may provide insights into the speed at which the person can translate visual information into a motor action. Hand Reaction Time may evaluate the potential efficiency of sensorimotor processing and the person's ability to initiate a manual response promptly. When performing the Go No-Go 3D, given the complexity of the test, the Hand Reaction Time will decrease. This particular behavior suggests that the primary motor cortex and the cerebellum are responding properly as the difficulty of the test increases. For example, if a person diagnosed with Parkinson perform the study, his/her hand reaction time will be slower and less flexible as the disabilities increase, and it can be inferred that the disease has a significant impact on hand speed fine-motor flexibility and on the primary motor cortex and the cerebellum. We may also apply Artificial Intelligence algorithms and conduct a stepwise approach wherein we combine eye movement and limb variables. We start by converting the input information to tabular datasets using biology-aware feature extraction methods and a diversity of normalization and aggregation methods. When selecting the AI algorithm we focus on robustness, selecting models that inherently address overfitting and class imbalance. Either in the data processing pipeline or in the model itself, we try to make the process as white-box and explainable as possible, allowing us to detect unexpected patterns and behaviours and to analyse them. We also perform a set of statistical analyses (as described above) to ensure cross-device compatibility, not only on the input data but also on the model results. Depending on dataset sizes, an out-of-bag cross-validation or a random-sample test set approach is used to evaluate the performance of the model. A variety of evaluation metrics may be used, which account for the general model performance but also for the inherent class imbalance and for the difference between Type-I and Type-II error costs found in the health field.

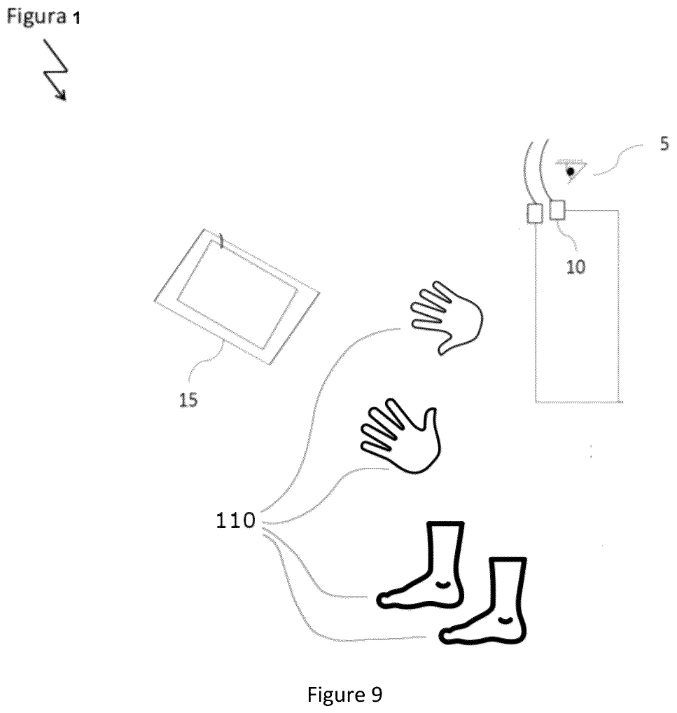

FIG. 9 shows a conceptual illustration of a system used to evaluate a person's [5] performance by applying a 3-Dimension Virtual Reality (3DVR) environment in combination with an embedded eye-tracking technology (ET) [10] and motions sensors to track the movement of limbs such as hands and feet [110] while the person performs well-defined activities [15]. The system shown in FIG. 9 may be used in the methods described below.

### 3D Virtual Reality and Eye-Tracking Technology in a Go No-GO Task

In order to quantify specific motor skills and cognitive processes, a method is presented to evaluate the performance in both healthy or non-healthy persons [5]. The method employs a 3-Dimension Virtual Reality (3DVR) environment such as described above and depicted in FIG. 9, which can be used to evaluate the person's performance, fine motor skills and cognitive capabilities by applying 3DVR, ET and limb movement tracking.

In accordance with the method, a person is requested to visualize objects on the VR screen while the eye movements are registered. Each object will have a defined feature such as colour, and it will move towards a person with a defined speed, acceleration and direction. For a number of repetitions, objects are presented in different zones to the person and the person is requested to visually observe the objects on the screen. The objects presented appear to move towards the subject in the 3D virtual environment and the objects will increase (or decrease) in apparent velocity when the person effectively virtually touches the correct objects by moving their limb towards the shape. Likewise, the object will decrease (or increase) in velocity when the person virtually touches the incorrect objects by moving their limbs.

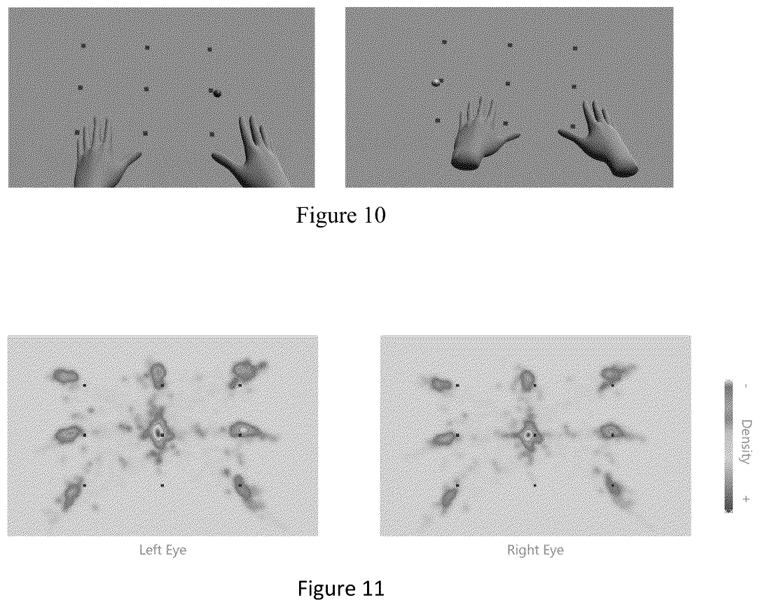

FIG. 10 shows an example of how objects may be positioned throughout the virtual environment and how objects and the person's hands may appear. In this example the objects may be presented in two different colors and appear to come from the background of the screen. The person is asked to touch the objects having one particular color. The person can use his or her right or left hand or right or left leg to touch the appropriate object, depending on whether the object is presented at the level of the hands or legs.

The method continues by measuring the saccades of the person in response to the presentation of the objects. In particular, the person is requested to look at the objects and touch them (or not touch them) in the virtual environment, and then the saccades are measured. These steps of requesting the person to visually observe the object and measuring saccades may be repeated multiple times.

Any of a variety of different requests may be made to the person to visualize and virtually touch (or not touch) objects on the VR screen while the eye movements are registered. For instance, the person may be requested to virtually touch objects having a certain feature (e.g., the color red) and not virtually touch objects having another feature (e.g., the color green). The aforementioned steps may be repeated for any number of instances of objects being presented, which may be presented at different speeds.

FIG. 11 is a graphical representation of an example of the density of eye movements recorded from the right and left eye while the person is performing the tasks touching the requested objects, presented as a heat-map.

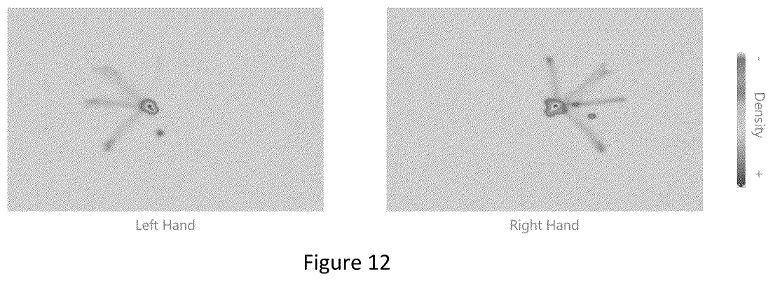

FIG. 12 is a graphical representation of the density of motor movements recorded of right and left-hand movements while the person is performing the tasks touching the requested objects, presented as a heat-map.

Based on the measurement of the eye and limb movements in response to the requests that are presented, any one or more of the following metrics may be calculated:
i. an inhibition process error (i.e. how many times the person touch the incorrect shapes); and
ii. an average saccadic latency, saccadic latency defined as an amount of time for said person to initiate a saccade to a new shape; and reporting one or more of
iii. a degree of compromise in processing speed with increased changes in the speed at which successive shapes are presented to the subject; and
iv. a degree of compromise in executive processes, with increased inhibition error. In some cases the method may include additional steps, including the step of making additional measurements during the step of presenting the objects to the person and requesting that the person look and touch (or not touch) the object. Illustrative examples of such additional measurements may include one or more of the following:
i. an amplitude of pupillary dilatation of the person;
ii. a number of fixations made by the person on said stimulus image; and
iii. the gaze duration by the person on the stimulus image;
iv. binocular disparity by the person while performing visual exploration and object visualization
v. object touched by person and fixations of where the visual stimulus was before
vi. number of consecutive object touched by person when performing a trial;
vii. Number of blinks coming from the left eye, the right eye or from both eyes.
viii. Time taken to visually detect objects (For a heat map of eye movements see FIG. 3).
ix. Time from the visualization of the object until the moment the person start to move the hands and/or feet.
x. Time since the person start to move the hands and/or feet up the time the person touches or tries to touch the object.
xi. Number of times the subject touch—or not—the virtual object (For a heat map of hand movements see FIG. 4).
xii. Optimal place for target visualization and places where visualizing targets is less efficient.
xii. Tracking Accuracy in maintaining visual focus on moving objects.
xiii. Hand-Reach Depth towards the objects during the act of touching.
xiv. Max Hand Velocity achieved by the hands during the movement towards the touched green objects.
xv. Dominant Hand Ratio of preferred hand usage during manual interactions.
xvi. Prediction Time, measuring the average time it takes for the person to anticipate and initiate a response following visual cues. xvii. Microsaccades; Factors of Form (FF):
1) HEWI: shows the microsacade's height/width relationship.
2) AREA: shows the area of the rectangle in which the microsaccade is inscribed.
3) LONG: is the longitude of the horizontal-vertical plane trajectory of the microsaccade.
4) ANG: is the sum of all the angles in the plane horizontal-vertical plane of the microsaccade.
5) AANG: is the sum of all the absolute values of angles in radians in the plane horizontal-vertical plane of the microsacaccade. These last two FF give an estimation of the microsaccadic trajectory regularity.
6) MOD and THETA: are the modulus and the angle of the polar coordinates of the sum of the cartesian coordinates. They give a spatial orientation of the microsaccade relative to the median of the fixation.
7) TIME: is the time duration in milliseconds of the microsaccade.
8) VMIN and VMAX: are the minimum and maximum velocities of the microsaccades in degrees per second.
9) Microsaccade rate: is the instantaneous rate in each time bin.
10) Directional congruency: is the congruency between the microsaccade direction and the location of the stimulus.

Other additional steps that may be taken while presenting the objects to the person and requesting that the person look and touch (or not touch) the object may include measuring the person's eye position coming from the left eye, the right eye or from both eyes (i.e., abscissa and ordinate coordinate) while performing visual exploration. The measurements may consider:
l. eye movements while visualizing and while touching objects;
ll. a total time when finalising the whole test; and
m. how many seconds are required for the person to visualize both correct and incorrect objects.

Yet another additional step that may be performed includes quantifying neurological processes and related cognitive activities when considering pupil size behavior and/or binocular disparity and/or micosaccade features and/or saccade behaviour and/or target touch rate and/or hands and feet movements and/or gazing and/or fixation duration and/or number of fixations and/or executive function performance in a 3DVR.

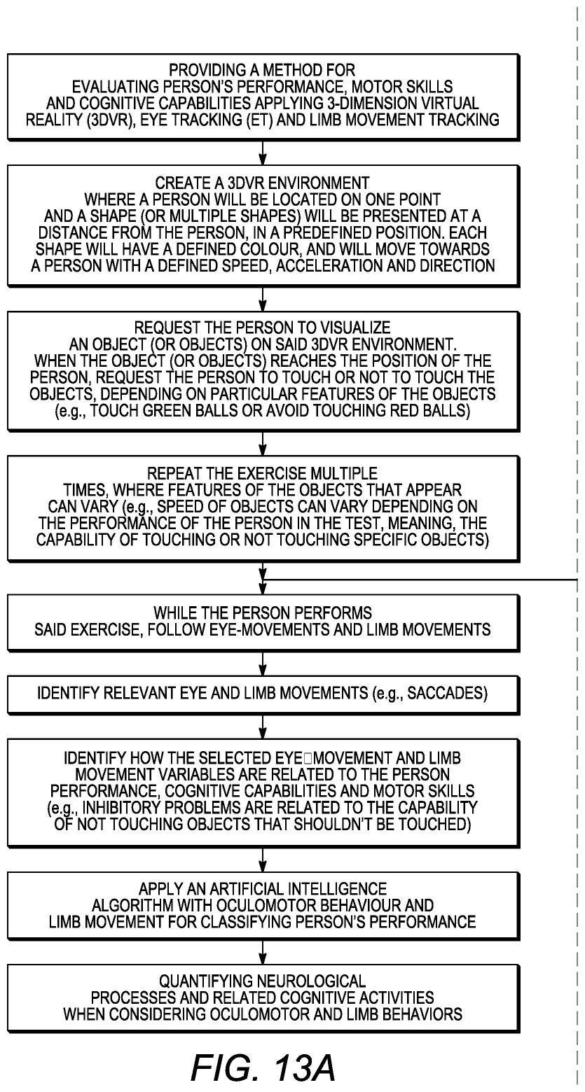

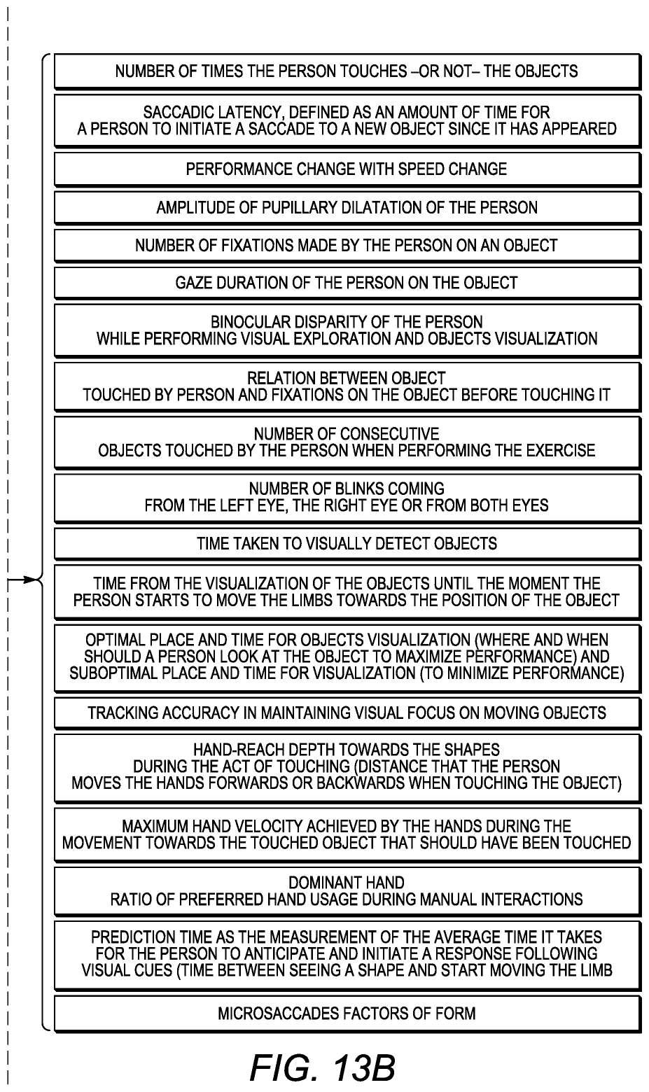

FIGS. 13A and 13B is a flowchart describing one example of the methods described herein for evaluating performance, motor skills and cognitive capabilities of a person.

1. A method for evaluating performance, motor skills and cognitive capabilities of a person, comprising:
requesting a person to perform a task, the task requesting the person to virtually touch specified virtual objects each having a different specified feature, the specified virtual objects being presented in a three-dimensional (3D) virtual reality environment, the virtual objects moving toward or away from the subject with a defined speed, acceleration and direction;
repeating the requesting by requesting the person to perform the task a plurality of times for different ones of the virtual objects having different specified features;
measuring eye movements and limb movements of the person while the subject is viewing the virtual objects and performing the tasks;
identifying selected ones of the measured eye and limb movements that are related to the performance, motor skills and cognitive capabilities of the person;
determining expected eye and limb movements of the person while the person is viewing the virtual objects and performing the tasks and comparing the expected eye and limb movements to the selected ones of the measured eye and limb movements to determine deviations therebetween; and
evaluating the performance, motor skills and cognitive capabilities of the person based on the deviations.

2. The method of claim 1 wherein the eye movements that are measured include at least one of a saccade amplitude, fixation duration and pupil behavior.

3. The method of claim 1 wherein the limb movements that are measured include a limb reaction time needed to perform a requested task.

4. The method of claim 1 wherein the different specified feature of the virtual objects is color (although not limited to).

5. The method of claim 1 wherein the evaluating includes determining metrics that include:
i. an inhibition process error (i.e. how many times the person touch the incorrect objects);
ii. an average saccadic latency, saccadic latency representing an amount of time needed for the person to initiate a saccade to view a successively viewed object.

6. The method of claim 1 wherein the evaluating further comprises determining (i) a degree of compromise in processing speed with increased changes in the speed at which successive objects are presented to the person (ii) a degree of compromise in executive processes, with increased inhibition error.

7. The method of claim 1 further comprising obtaining one or more additional measurements while the person is viewing the virtual objects and performing the tasks, the one or more additional measurements being selected from the group consisting of:
i. an amplitude of pupillary dilatation of the person;
ii. a number of fixations made by the person on said stimulus image;
iii. the gaze duration by the person on the stimulus image;
iv. binocular disparity by the person while performing visual exploration and objects visualization;
v. target touched by person and fixations of where the visual stimulus was before;
vi. number of consecutive object touched by person when performing a trial;
vii. Number of blinks coming from the left eye, the right eye or from both eyes;
viii. Time taken to visually detect objects;
x. Time since the person start to move the hands and/or feet up the time the person touches or tries to touch the object;
xi. Number of times the subject touch—or not—the virtual objects;
xii. Optimal place for target visualization and places where visualizing objects is less efficient.
xii. Tracking Accuracy in maintaining visual focus on moving objects;
xiii. Hand-Reach Depth towards the objects during the act of touching;
xiv. Max Hand Velocity achieved by the hands during the movement towards the touched green objects;
xv. Dominant Hand Ratio of preferred hand usage during manual interactions;
xvi. Prediction Time, measuring the average time it takes for the person to anticipate and initiate a response following visual cues; and
xii. Microsaccades.

8. A system for evaluating performance, motor skills and cognitive capabilities of a person, comprising:
a three-dimensional (3D) virtual reality device configured to establish a 3D virtual reality environment in which a plurality of virtual objects is presented to the person, the objects having at least one feature that differs from one another, the objects moving toward or away from the person with a defined speed, acceleration and direction;
an eye-tracker configured to measure eye movements of the person while the person is viewing the virtual objects and performing requested tasks, the requested tasks including multiple requests requesting the person to virtually touch specified virtual objects each having one of the specified features;
one or more motion sensors configured to measure limb movements of the person while the person performs the requested tasks;
a processor configured to receive data from the 3D virtual reality device, the eye-tracker and the one or more motion sensors while the person is performing the requested tasks and being further configured to (i) identify selected ones of the measured eye and limb movements that are related to the performance, motor skills and cognitive capabilities of the person; (ii) determine expected eye and limb movements of the person while the person is viewing the virtual objects and performing the requested tasks and comparing the expected eye and limb movements to the selected ones of the measured eye and limb movements to determine deviations therebetween; and (iii) evaluate the performance, motor skills and cognitive capabilities of the person based on the deviations.
